SEQUENCE LISTING (8 sequences)

SEQ ID NO: 1 - DNA, 9204 bp, Artificial Sequence
  Synthetic Sequence pBC.1 (pUMVC4a-HER2ECD_Mucin1_HER2ICD)
ccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcggccaccatggagctggcggccttgtgccgctgggggctcctcctcgccctcttgccccccggagccgcgagcacccaagtgtgcaccggcacagacatgaagctgcggctccctgccagtcccgagacccacctggacatgctccgccacctctaccagggctgccaggtggtgcagggaaacctggaactcacctacctgcccaccaatgccagcctgtccttcctgcaggatatccaggaggtgcagggctacgtgctcatcgctcacaaccaagtgaggcaggtcccactgcagaggctgcggattgtgcgaggcacccagctctttgaggacaactatgccctggccgtgctagacaatggagacccgctgaacaataccacccctgtcacaggggcctccccaggaggcctgcgggagctgcagcttcgaagcctcacagagatcttgaaaggaggggtcttgatccagcggaacccccagctctgctaccaggacacgattttgtggaaggacatcttccacaagaacaaccagctggctctcacactgatagacaccaaccgctctcgggcctgccacccctgttctccgatgtgtaagggctcccgctgctggggagagagttctgaggattgtcagagcctgacgcgcactgtctgtgccggtggctgtgcccgctgcaaggggccactgcccactgactgctgccatgagcagtgtgctgccggctgcacgggccccaagcactctgactgcctggcctgcctccacttcaaccacagtggcatctgtgagctgcactgcccagccctggtcacctacaacacagacacgtttgagtccatgcccaatcccgagggccggtatacattcggcgccagctgtgtgactgcctgtccctacaactacctttctacggacgtgggatcctgcaccctcgtctgccccctgcacaaccaagaggtgacagcagaggatggaacacagcggtgtgagaagtgcagcaagccctgtgcccgagtgtgctatggtctgggcatggagcacttgcgagaggtgagggcagttaccagtgccaatatccaggagtttgctggctgcaagaagatctttgggagcctggcatttctgccggagagctttgatggggacccagcctccaacactgccccgctccagccagagcagctccaagtgtttgagactctggaagagatcacaggttacctatacatctcagcatggccggacagcctgcctgacctcagcgtcttccagaacctgcaagtaatccggggacgaattctgcacaatggcgcctactcgctgaccctgcaagggctgggcatcagctggctggggctgcgctcactgagggaactgggcagtggactggccctcatccaccataacacccacctctgcttcgtgcacacggtgccctgggaccagctctttcggaacccgcaccaagctctgctccacactgccaaccggccagaggacgagtgtgtgggcgagggcctggcctgccaccagctgtgcgcccgagggcactgctggggtccagggcccacccagtgtgtcaactgcagccagttccttcggggccaggagtgcgtggaggaatgccgagtactgcaggggctccccagggagtatgtgaatgccaggcactgtttgccgtgccaccctgagtgtcagccccagaatggctcagtgacctgttttggaccggaggctgaccagtgtgtggcctgtgcccactataaggaccctcccttctgcgtggcccgctgccccagcggtgtgaaacctgacctctcctacatgcccatctggaagtttccagatgaggagggcgcatgccagccttgccccatcaactgcacccactcctgtgtggacctggatgacaagggctgccccgccgagcagagagccagccctctgacgggcagcggcgccacaaacttctctctgctaaagcaagcaggtgatgttgaagaaaaccccgggcctacaccgggcacccagtctcctttcttcctgctgctgctcctcacagtgcttacagttgttacgggttctggtcatgcaagctctaccccaggtggagaaaaggagacttcggctacccagagaagttcagtgcccagctctactgagaagaatgctccggacaccaggccggccccgggctccaccgcccccccagcccatggtgtcacctcgttgtctactggggtctctttctttttcctgtcttttcacatttcaaacctccagtttaattcctctctggaagatcccagcaccgactactaccaagagctgcagagagacatttctgaaatgtttttgcagatttataaacaagggggttttctgggcctctccaatattaagttcaggccaggatctgtggtggtacaattgactctggccttccgagaaggtaccatcaatgtccacgacgtggagacacagttcaatcagtataaaacggaagcagcctctcgatataacctgacgatctcagacgtcagcgtgagtgatgtgccatttcctttctctgcccagtctggggctggggtgccaggctggggcatcgcgctgctggtgctggtctgtgttctggttgcgctggccattgtctatctcattgccttggctgtctgtcagtgccgccgaaagaactacgggcagctggacatctttccagcccgggatacctaccatcctatgagcgagtaccccacctaccacacccatgggcgctatgtgccccctagcagtaccgatcgtagcccctatgagaaggtttctgcaggtaatggtggcagcagcctctcttacacaaacccagcagtggcagccacttctgccaacttgggctccggcgagggcaggggaagtcttctaacatgcggggacgtggaggaaaatcccggcccagctttcctctggctcctctcctgctgggccctcctgggtaccaccttcggcaagcgacggcagcagaagatccggaagtacacgatgcggagactgctgcaggaaacggagctggtggagccgctgacacctagcggagcgatgcccaaccaggcgcagatgcggatcctgaaagagacggagctgaggaaggtgaaggtgcttggatctggcgcttttggcacagtctacaagggcatctggatccctgatggggagaatgtgaaaattccagtggccatcaaagtgttgagggaaaacacatcccccaaagccaacaaagaaatcttagacgaagcatacgtgatggctggtgtgggctccccatatgtctcccgccttctgggcatctgcctgacatccacggtgcagctggtgacacagcttatgccctatggctgcctcttagaccatgtccgggaaaaccgcggacgcctgggctcccaggacctgctgaactggtgtatgcagattgccaaggggatgagctacctggaggatgtgcggctcgtacacagggacttggccgctcggaacgtgctggtcaagagtcccaaccatgtcaaaattacagacttcgggctggctcggctgctggacattgacgagacagagtaccatgcagatgggggcaaggtgcccatcaagtggatggcgctggagtccattctccgccggcggttcacccaccagagtgatgtgtggagttatggtgtgactgtgtgggagctgatgacttttggggccaaaccttacgatgggatcccagcccgggagatccctgacctgctggaaaagggggagcggctgccccagccccccatctgcaccattgatgtctacatgatcatggtcaaatgttggatgattgactctgaatgtcggccaagattccgggagttggtgtctgaattctcccgcatggccagggacccccagcgctttgtggtcatccagaatgaggacttgggcccagccagtcccttggacagcaccttctaccgctcactgctggaggacgatgacatgggggacctggtggatgctgaggagtatctggtaccccagcagggcttcttctgtccagaccctgccccgggcgctgggggcatggtccaccacaggcaccgcagctcatctaccaggagtggcggtggggacctgacactagggctggagccctctgaagaggaggcccccaggtctccactggcaccctccgaaggggctggctccgatgtatttgatggtgacctgggaatgggggcagccaaggggctgcaaagcctccccacacatgaccccagccctctacagcggtacagtgaggaccccacagtacccctgccctctgagactgatggctacgttgcccccctgacctgcagcccccagcctgaatatgtgaaccagccagatgttcggccccagcccccttcgccccgagagggccctctgcctgctgcccgacctgctggtgccactctggaaaggcccaagactctctccccagggaagaatggggtcgtcaaagacgtttttgcctttgggggtgccgtggagaaccccgagtacttgacaccccagggaggagctgcccctcagccccaccctcctcctgccttcagcccagccttcgacaacctctattactgggaccaggacccaccagagcggggggctccacccagcaccttcaaagggacacctacggcagagaacccagagtacctgggtctggacgtgccagtgtgaagcggccgcggatccagatctttttccctcgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaa

SEQ ID NO: 2 - protein, 1583 aa, Artificial Sequence
  Synthetic Sequence Antigen protein sequence encoded by pBC.1 (pUMVC4a-HER2ECD_Mucin1_HER2ICD)
MELAALCRWGLLLALLPPGAASTQVCTGTDMKLRLPATPETHLDMLRHLYQGCQVVQGNLELTYLPTNASLSFLQDIQEVQGYVLIAHNQVRQVPLQRLRIVRGTQLFEDNYALAVLDNGDPLNNTTPVTGASPGGLRELQLRSLTEILKGGVLIQRNPQLCYQDTILWKDIFHKNNQLALTLIDTNRSRACHPCSPMCKGSRCWGESSEDCQSLTRTVCAGGCARCKGPLPTDCCHEQCAAGCTGPKHSDCLACLHFNHSGICELHCPALVTYNTDTFESMPNPEGRYTFGASCVTACPYNYLSTDVGSCTLVCPLHNQEVTAEDGTQRCEKCSKPCARVCYGLGMEHLREVRAVTSANIQELAGCKKIFGSLAFLPESFDGDPASNTAPLQPEQLQVFETLEEITGYLYISAWPDSLPDLSVFQNLQVIRGRILHNGAYSLTLQGLGISWLGLRSLRELGSGLALIHHNTHLCFVHTVPWDQLFRNPHQALLHTANRPEDECVGEGLACHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLTGSGATNFSLLKQAGDVEENPGPTPGTQSPFFLLLLLTVLTVVTGSGHASSTPGGEKETSATQRSSVPSSTEKNAPDTRPAPGSTAPPAHGVTSLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAATSANLGSGEGRGSLLTCGDVEENPGPAFLWLLSCWALLGTTFGKRRQQKIRKYTMRRLLQETELVEPLTPSGAMPNQAQMRILKETELRKVKVLGSGAFGTVYKGIWIPDGENVKIPVAIKVLRENTSPKANKEILDEAYVMAGVGSPYVSRLLGICLTSTVQLVTQLMPYGCLLDHVRENRGRLGSQDLLNWCMQIAKGMSYLEDVRLVHRDLAARNVLVKSPNHVKITDFGLARLLDIDETEYHADGGKVPIKWMALESILRRRFTHQSDVWSYGVTVWELMTFGAKPYDGIPAREIPDLLEKGERLPQPPICTIDVYMIMVKCWMIDSECRPRFRELVSEFSRMARDPQRFVVIQNEDLGPASPLDSTFYRSLLEDDDMGDLVDAEEYLVPQQGFFCPDPAPGAGGMVHHRHRSSSTRSGGGDLTLGLEPSEEEAPRSPLAPSEGAGSDVFDGDLGMGAAKGLQSLPTHDPSPLQRYSEDPTVPLPSETDGYVAPLTCSPQPEYVNQPDVRPQPPSPREGPLPAARPAGATLERPKTLSPGKNGVVKDVFAFGGAVENPEYLTPQGGAAPQPHPPPAFSPAFDNLYYWDQDPPERGAPPSTFKGTPTAENPEYLGLDVPV

SEQ ID NO: 3 - DNA, 9818 bp, Artificial Sequence
  Synthetic Sequence mvaBC.1 (pMVa-mH5_HER2ECD_Mucin1_HER2ICD)
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtaggatcgcaagaatttatttctaaatctatgagagatcaacgaaaaggaagagtaggaagagttaatcctgggacatacgtatatttctatgatctgtcttatatgaagtctatacagcgaatagattcagaatttctacataattatatattgtacgctaataagtttaatctaacactccccgaagatttgtttataatccctacaaatttggatattctatggcgtacaaaggaatatatagactcgttcgatattagtacagaaacatggaataaattattatccaattattatatgaagatgatagagtatgctaaactttatgtactaagtcctattctcgctgaggagttggataattttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaacggccgtttcattttgtttttttctatgctataaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgttgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggttgagaggacgggagaattaactagtattgtacaagaagccattttatctctaaatttacgaattaagattttaaattttaaacataaagatgatgatacgtatatacacttttgtaaaatattattcggtgtctataacggaacaaacgctactatatattatcatagacctctaacgggatatatgaatatgatttcagatactatatttgttcctgtagataataactaaaaggtcaagcttggtacccaacctaaaaattgaaaataaatacaaaggttcttgagggttgtgttaaattgaaagcgagaaataatcataaataagcccaaaccatggaactagcagctctatgtagatggggactactactagctctactaccacctggtgctgcttctacacaagtctgtactggaaccgacatgaagctaagactaccagctactccagaaacgcacctagatatgctaagacacctataccaaggatgccaggtcgtacagggaaacctagaactaacatacctaccgaccaacgcctccttgtccttcctacaagatatccaagaagtccagggatacgtcctaatcgctcacaatcaagttagacaagtcccactacagagactaagaatcgtcagaggaacccagctattcgaggataattatgctctagcagtcctagacaacggtgatccattgaacaacacaacaccagtcacaggtgctagtccaggtggactaagagaactacagctaagatccctaaccgagatcctaaaaggtggtgtcctaatccaaagaaacccacagctatgttaccaggacaccatcctatggaaggacatcttccacaagaacaaccagctagctctaacactaatcgacaccaatagatctagagcgtgtcatccgtgttctccgatgtgtaaaggatctagatgttggggagagtcgtccgaggattgtcagtctctaacaagaaccgtatgtgctggtggatgtgcgagatgtaaaggaccattaccgacagattgctgtcacgaacaatgtgctgcaggatgtacaggaccgaaacactctgattgtctagcctgcctacacttcaaccactctggaatctgtgaactacattgtccagcactagtaacctacaacaccgatacctttgagtccatgccaaatccagagggaagatacacattcggagcgtcttgtgtaacagcgtgtccgtacaactacctatccacagatgtaggatcttgcaccctagtttgtccgttgcataatcaagaagtaacagctgaggacggaacccagagatgtgaaaaatgttctaaaccgtgtgcgagagtctgctacggattaggaatggaacacctaagagaagtcagagcagttacctctgcgaacatccaagaattagctggatgcaagaagatctttggatctctagctttcctaccagaatcctttgatggtgatcctgcttctaatactgctccactacaacctgaacagctacaggtgttcgaaaccctagaagagatcactggatacttgtacatttctgcgtggccagattccttgcctgacctatctgtattccagaacctacaagtcatcagaggtagaatcctacacaacggtgcctactcgctaacgttacaaggactaggaatctcctggctaggactaagatccttgagagagctaggatctggacttgcgttaatccatcataacacccacctatgtttcgtccataccgtaccatgggaccagttgttcagaaatccacatcaagcgttactacacacagcgaatagaccagaagatgaatgtgtcggagaaggactagcctgtcatcagttatgtgctagaggacactgttggggaccaggaccaacacaatgtgtaaactgctcccagttcctaagaggacaagagtgtgtcgaagagtgcagagtactacaaggattaccgagagaatacgtcaatgcgagacattgtctaccatgtcacccagaatgtcaaccacagaacggatctgtaacatgttttggaccagaagcagatcagtgtgtagcttgtgctcattataaagatccaccgttttgtgtagcgagatgtccatctggtgtaaagcctgatttgtcctatatgccaatctggaagttcccagatgaagagggagcttgtcaaccttgtccgatcaactgtacccattcctgtgtcgatctagacgataagggatgtccagcagaacaaagagcttctccattaactggatctggtgccaccaacttctcgctattgaaacaagcgggagatgtcgaagaaaatccaggacctactccaggaacacagtctccattcttcttgctactactattgaccgtcctaaccgtcgtaactggttctggacatgcatcttctacaccaggtggagagaaagaaacatctgcgactcagagatcctccgtaccttcctctactgaaaaaaacgctccagatacaagacctgctccaggatctacagctccaccagctcatggtgtaacatctctatctactggtgtctcgttcttcttcctatcgttccacatctccaacctacagttcaactcgtccttggaagatccgtccaccgattactaccaagagttgcagagagacatctccgaaatgttcctacagatctacaagcaaggaggattcttgggtctatccaacatcaagttcagaccaggatctgtagtcgtccaactaacactagcgttcagagaaggtactatcaacgtccacgatgtcgaaacccagttcaaccagtataagacagaagcagcgtcgagatacaacctaaccatctctgatgtctccgtgtccgatgtaccgtttccattttctgctcaatctggtgctggtgtaccaggatggggaattgctctactagtcctagtatgtgtcctagtcgctctagcgatcgtatatctaattgcgttggcagtctgccaatgtagaagaaagaactacggacagctagatatcttcccagctagagatacttaccatccgatgtctgaatacccgacctaccatacacacggaagatatgtaccaccgtcctccacagatagatcaccatacgaaaaagtctctgctggaaatggtggttcctcgctatcttatacaaatccagctgtagctgcgacgtctgcgaatctaggaagtggagaaggaagaggatccttgctaacctgtggagatgtagaggaaaaccctggtccagctttcttgtggctactatcttgttgggcactattgggaacaaccttcggaaagagaagacagcagaagatcagaaagtacacgatgagaagattgctacaagaaaccgagctagtcgaaccattaacaccttctggtgctatgcctaatcaagctcagatgagaatcttgaaagagacagagttgagaaaagtcaaggtcctaggttctggtgccttcggaacagtatacaaaggaatttggattccagacggagagaacgtcaaaatcccagtagcgatcaaggtgctaagagagaacacttctccgaaagcgaacaaagaaatcctagatgaagcgtacgtaatggcaggtgttggatctccatatgtctccagactacttggaatctgcctaacctctaccgtacagctagtcacacaactaatgccgtacggatgtctactagaccacgtcagagaaaacagaggaagattgggatcgcaggacctattgaactggtgtatgcaaatcgcgaagggaatgtcctacctagaggacgttagactagtccatagagatctagctgcgagaaacgtactagttaagtctccgaaccacgtcaagatcaccgattttggactagcgagactactagacatcgacgaaacagaataccacgctgatggtggaaaagtaccgatcaaatggatggctctagagtccatcctaagaagaagattcacccaccagtccgacgtatggtcttacggtgtaacagtatgggagctaatgacctttggagcgaagccgtatgatggaataccagcgagagaaatcccagacttgctagaaaagggagaaagattgccacagccaccgatctgtaccatcgacgtctacatgattatggtcaagtgctggatgatcgattccgagtgtagaccgagattcagagagcttgtctcggagttttctagaatggcaagagatccacagagattcgtagtaatccagaacgaggatctaggaccagcctctccactagactctaccttctatagatccctattggaggacgacgacatgggagacctagtagatgctgaagaatacctagtaccacagcagggattcttctgtcctgatccagctccaggtgcaggtggaatggtacatcatagacacagatcctcgagtacgagatctggtggtggagatctaaccctaggattggaaccgtctgaagaagaagctccaagatctccattagctccatctgaaggtgctggatctgatgtattcgacggagatctaggaatgggagctgcgaaaggactacaatctctaccgacacatgatccgtctccgctacaaagatattctgaagatccgacagtaccgttgccgtctgaaacagatggatatgtagcaccactaacatgttctcctcaacctgaatatgtaaaccagccagacgtcagaccacaaccaccatctccaagagaaggacctttaccagctgctagacctgctggtgcaacactagaaagaccgaaaactttgtctccaggaaagaacggtgtagtcaaggatgtatttgcgttcggaggtgcagtagaaaacccagaatatctaactccacaaggtggtgcagctccacaacctcatccacctccagcattttctccagcgttcgacaacctatattactgggatcaagatcctccagaaagaggtgctccaccatctacttttaagggaacaccgacagctgagaatccagagtacttgggactagatgtacctgtctaaggatcctagttaattaaaatcaaactctaatgaccacatctttttttagagatgaaaaattttccacatctccttttgtagacacgactaaacattttgcagaaaaaagtttattagtgtttagataatcgtatacttcatcagtgtagatagtaaatgtgaacagataaaaggtattcttgctcaatagattggtaaattccatagaatatattaatcctttcttcttgagatcccacatcatttcaaccagagacgttttatccaatgatttacctcgtactataccacatacaaaactagattttgcagtgacgtcgtatctggtattcctaccaaacaaaattttacttttagttcttttagaaaattctaaggtagaatctctatttgccaatatgtcatctatggaattaccactagcaaaaaatgatagaaatatatattgatacatcgcagctggttttgatctactatactttaaaaacgaatcagattccataattgcctgtatatcatcagctgaaaaactatgttttacacgtattccttcggcatttctttttaatgatatatcttgtttagacaatgataaagttatcatgtccatgagagacgcgtctccgtatcgtataaatatttcattagatgttagacgcttcattaggggtatacttctataaggtttcttaatcagtccatcattggttgcgtcaagaactactatcggatgttgttaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

SEQ ID NO: 4 - protein, 1583 aa, Artificial Sequence; identical to SEQ ID NO: 2
  Synthetic Sequence Antigen protein sequence encoded by mvaBC.1 (pMVa-mH5_HER2ECD_Mucin1_HER2ICD)

SEQ ID NO: 5 - DNA, 9767 bp, Artificial Sequence
  Synthetic Sequence pBC.2 (pUMVC4a-Mamma_Magea3_Survivin_Tert)
tccggtgagaatggcaaaagcttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgttttcccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaatcgatagattgtcgcacctgattgcccgacattatcgcgagcccatttatacccatataaatcagcatccatgttggaatttaatcgcggcctcgagcaagacgtttcccgttgaatatggctcataacaccccttgtattactgtttatgtaagcagacagttttattgttcatgatgatatatttttatcttgtgcaatgtaacatcagagattttgagacacaacgtggctttccccccccccccattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcagattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactccaacggtggagggcagtgtagtctgagcagtactcgttgctgccgcgcgcgccaccagacataatagctgacagactaacagactgttcctttccatgggtcttttctgcagtcaccgtcgtcgacggtatcgataagcttgatatcggccaccatgaagttgctgatggtcctcatgctggcggccctctcccagcactgctacgcaggctctggctgccccttattggagaatgtgatttccaagacaatcaatccacaagtgtctaagactgaatacaaagaacttcttcaagagttcatagacgacaatgccactacaaatgccatagatgaattgaaggaatgttttcttaaccaaacggatgaaactctgagcaatgttgaggtgtttatgcaattaatatatgacagcagtctttgtgatttatttggatccggagctaccaattttagcctgctgaaacaggccggcgacgtggaagagaatcctggacctgctttcctctggctcctctcctgctgggccctcctgggtaccaccttcggccctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagggcagcggcgccacaaacttctctctgctaaagcaagcaggtgatgttgaagaaaaccccgggcctaatctacttctgatccttacctttgttgcagctgctgttgctggtgccccgacgttgccccctgcctggcagccctttctcaaggaccaccgcatctctacattcaagaactggcccttcttggagggctgcgcctgcaccccggagcggatggccgaggctggcttcatccactgccccactgagaacgagccagacttggcccagtgtttcttctgcttcaaggagctggaaggctgggagccagatgacgaccccatagaggaacataaaaagcattcgtccggttgcgctttcctttctgtcaagaagcagtttgaagaattaacccttggtgaatttttgaaactggacagagaaagagccaagaacaaaattgcaaaggaaaccaacaataagaagaaagaatttgaggaaactgcggagaaagtgcgccgtgccatcgagcagctggctgccatggatggctccggcgagggcaggggaagtcttctaacatgcggggacgtggaggaaaatcccggcccaaagtgggtaacctttatttcccttctttttctctttagctcggcttattccagccactaccgcgaggtgctgccgctggccacgttcgtgcggcgcctggggccccagggctggcggctggtgcagcgcggggacccggcggctttccgcgcgctggtggcccagtgcctggtgtgcgtgccctgggacgcacggccgccccccgccgccccctccttccgccaggtgtcctgcctgaaggagctggtggcccgagtgctgcagaggctgtgcgagcgcggcgcgaagaacgtgctggccttcggcttcgcgctgctggacggggcccgcgggggcccccccgaggccttcaccaccagcgtgcgcagctacctgcccaacacggtgaccgacgcactgcgggggagcggggcgtgggggctgctgctgcgccgcgtgggcgacgacgtgctggttcacctgctggcacgctgcgcgctctttgtgctggtggctcccagctgcgcctaccaggtgtgcgggccgccgctgtaccagctcggcgctgccactcaggcccggcccccgccacacgctagtggaccccgaaggcgtctgggatgcgaacgggcctggaaccatagcgtcagggaggccggggtccccctgggcctgccagccccgggtgcgaggaggcgcgggggcagtgccagccgaagtctgccgttgcccaagaggcccaggcgtggcgctgcccctgagccggagcggacgcccgttgggcaggggtcctgggcccacccgggcaggacgcgtggaccgagtgaccgtggtttctgtgtggtgtcacctgccagacccgccgaagaagccacctctttggagggtgcgctctctggcacgcgccactcccacccatccgtgggccgccagcaccacgcgggccccccatccacatcgcggccaccacgtccctgggacacgccttgtcccccggtgtacgccgagaccaagcacttcctctactcctcaggcgacaaggagcagctgcggccctccttcctactcagctctctgaggcccagcctgactggcgctcggaggctcgtggagaccatctttctgggttccaggccctggatgccagggactccccgcaggttgccccgcctgccccagcgctactggcaaatgcggcccctgtttctggagctgcttgggaaccacgcgcagtgcccctacggggtgctcctcaagacgcactgcccgctgcgagctgcggtcaccccagcagccggtgtctgtgcccgggagaagccccagggctctgtggcggcccccgaggaggaggacacagacccccgtcgcctggtgcagctgctccgccagcacagcagcccctggcaggtgtacggcttcgtgcgggcctgcctgcgccggctggtgcccccaggcctctggggctccaggcacaacgaacgccgcttcctcaggaacaccaagaagttcatctccctggggaagcatgccaagctctcgctgcaggagctgacgtggaagatgagcgtgcgggactgcgcttggctgcgcaggagcccaggggttggctgtgttccggccgcagagcaccgtctgcgtgaggagatcctggccaagttcctgcactggctgatgagtgtgtacgtcgtcgagctgctcaggtctttcttttatgtcacggagaccacgtttcaaaagaacaggctctttttctaccggaagagtgtctggagcaagttgcaaagcattggaatcagacagcacttgaagagggtgcagctgcgggagctgtcggaagcagaggtcaggcagcatcgggaagccaggcccgccctgctgacgtccagactccgcttcatccccaagcctgacgggctgcggccgattgtgaacatggactacgtcgtgggagccagaacgttccgcagagaaaagagggccgagcgtctcacctcgagggtgaaggcactgttcagcgtgctcaactacgagcgggcgcggcgccccggcctcctgggcgcctctgtgctgggcctggacgatatccacagggcctggcgcaccttcgtgctgcgtgtgcgggcccaggacccgccgcctgagctgtactttgtcaaggtggatgtgacgggcgcgtacgacaccatcccccaggacaggctcacggaggtcatcgccagcatcatcaaaccccagaacacgtactgcgtgcgtcggtatgccgtggtccagaaggccgcccatgggcacgtccgcaaggccttcaagagccacgtctctaccttgacagacctccagccgtacatgcgacagttcgtggctcacctgcaggagaccagcccgctgagggatgccgtcgtcatcgagcagagctcctccctgaatgaggccagcagtggcctcttcgacgtcttcctacgcttcatgtgccaccacgccgtgcgcatcaggggcaagtcctacgtccagtgccaggggatcccgcagggctccatcctctccacgctgctctgcagcctgtgctacggcgacatggagaacaagctgtttgcggggattcggcgggacgggctgctcctgcgtttggtggatgatttcttgttggtgacacctcacctcacccacgcgaaaaccttcctcaggaccctggtccgaggtgtccctgagtatggctgcgtggtgaacttgcggaagacagtggtgaacttccctgtagaagacgaggccctgggtggcacggcttttgttcagatgccggcccacggcctattcccctggtgcggcctgctgctggatacccggaccctggaggtgcagagcgactactccagctatgcccggacctccatcagagccagtctcaccttcaaccgcggcttcaaggctgggaggaacatgcgtcgcaaactctttggggtcttgcggctgaagtgtcacagcctgtttctggatttgcaggtgaacagcctccagacggtgtgcaccaacatctacaagatcctcctgctgcaggcgtacaggtttcacgcatgtgtgctgcagctcccatttcatcagcaagtttggaagaaccccacatttttcctgcgcgtcatctctgacacggcctccctctgctactccatcctgaaagccaagaacgcagggatgtcgctgggggccaagggcgccgccggccctctgccctccgaggccgtgcagtggctgtgccaccaagcattcctgctcaagctgactcgacaccgtgtcacctacgtgccactcctggggtcactcaggacagcccagacgcagctgagtcggaagctcccggggacgacgctgactgccctggaggccgcagccaacccggcactgccctcagacttcaagaccatcctggactgatttttccctctgccaaaaattatggggacatcatgaagccccttgagcatctgacttctggctaataaaggaaatttatttcattgcaatagtgtgttggaattttttgtgtctctcactcggaaggacatatgggagggcaaatcatttaaaacatcagaatcagtatttggtttagagtttggcaacatatgccattcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccggggggggggggcgctgaggtctgcctcgtgaagaaggtgttgctgactcataccagggcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccgccgtcccgtcaagtcagcgtaatgctctgccagtgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgatccgactcgtccaacatcaatacaacctattaatttcccctcgtcaaaaataaggttatcaagtgagaaatcaccatgagtgacgactgaa

SEQ ID NO: 6 - protein, 1777 aa, Artificial Sequence
  Synthetic Sequence Antigen protein sequence encoded by pBC.2 (pUMVC4a-Mamma_Magea3_Survivin_Tert)
MKLLMVLMLAALSQHCYAGSGCPLLENVISKTINPQVSKTEYKELLQEFIDDNATTNAIDELKECFLNQTDETLSNVEVFMQLIYDSSLCDLFGSGATNFSLLKQAGDVEENPGPAFLWLLSCWALLGTTFGPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEEGSGATNFSLLKQAGDVEENPGPNLLLILTFVAAAVAGAPTLPPAWQPFLKDHRISTFKNWPFLEGCACTPERMAEAGFIHCPTENEPDLAQCFFCFKELEGWEPDDDPIEEHKKHSSGCAFLSVKKQFEELTLGEFLKLDRERAKNKIAKETNNKKKEFEETAEKVRRAIEQLAAMDGSGEGRGSLLTCGDVEENPGPKWVTFISLLFLFSSAYSSHYREVLPLATFVRRLGPQGWRLVQRGDPAAFRALVAQCLVCVPWDARPPPAAPSFRQVSCLKELVARVLQRLCERGAKNVLAFGFALLDGARGGPPEAFTTSVRSYLPNTVTDALRGSGAWGLLLRRVGDDVLVHLLARCALFVLVAPSCAYQVCGPPLYQLGAATQARPPPHASGPRRRLGCERAWNHSVREAGVPLGLPAPGARRRGGSASRSLPLPKRPRRGAAPEPERTPVGQGSWAHPGRTRGPSDRGFCVVSPARPAEEATSLEGALSGTRHSHPSVGRQHHAGPPSTSRPPRPWDTPCPPVYAETKHFLYSSGDKEQLRPSFLLSSLRPSLTGARRLVETIFLGSRPWMPGTPRRLPRLPQRYWQMRPLFLELLGNHAQCPYGVLLKTHCPLRAAVTPAAGVCAREKPQGSVAAPEEEDTDPRRLVQLLRQHSSPWQVYGFVRACLRRLVPPGLWGSRHNERRFLRNTKKFISLGKHAKLSLQELTWKMSVRDCAWLRRSPGVGCVPAAEHRLREEILAKFLHWLMSVYVVELLRSFFYVTETTFQKNRLFFYRKSVWSKLQSIGIRQHLKRVQLRELSEAEVRQHREARPALLTSRLRFIPKPDGLRPIVNMDYVVGARTFRREKRAERLTSRVKALFSVLNYERARRPGLLGASVLGLDDIHRAWRTFVLRVRAQDPPPELYFVKVDVTGAYDTIPQDRLTEVIASIIKPQNTYCVRRYAVVQKAAHGHVRKAFKSHVSTLTDLQPYMRQFVAHLQETSPLRDAVVIEQSSSLNEASSGLFDVFLRFMCHHAVRIRGKSYVQCQGIPQGSILSTLLCSLCYGDMENKLFAGIRRDGLLLRLVDDFLLVTPHLTHAKTFLRTLVRGVPEYGCVVNLRKTVVNFPVEDEALGGTAFVQMPAHGLFPWCGLLLDTRTLEVQSDYSSYARTSIRASLTFNRGFKAGRNMRRKLFGVLRLKCHSLFLDLQVNSLQTVCTNIYKILLLQAYRFHACVLQLPFHQQVWKNPTFFLRVISDTASLCYSILKAKNAGMSLGAKGAAGPLPSEAVQWLCHQAFLLKLTRHRVTYVPLLGSLRTAQTQLSRKLPGTTLTALEAAANPALPSDFKTILD

SEQ ID NO: 7 - protein, 10399 aa, Artificial Sequence
  Synthetic Sequence mvaBC.2 (pMVa-mH5_Mamma_Magea3_Survivin_Tert)
CGCGCGTTTCGGTGATGACGGTGAAAACCTCTGACACATGCAGCTCCCGGAGACGGTCACAGCTTGTCTGTAAGCGGATGCCGGGAGCAGACAAGCCCGTCAGGGCGCGTCAGCGGGTGTTGGCGGGTGTCGGGGCTGGCTTAACTATGCGGCATCAGAGCAGATTGTACTGAGAGTGCACCATATGCGGTGTGAAATACCGCACAGATGCGTAAGGAGAAAATACCGCATCAGGCGCCATTCGCCATTCAGGCTGCGCAACTGTTGGGAAGGGCGATCGGTGCGGGCCTCTTCGCTATTACGCCAGCTGGCGAAAGGGGGATGTGCTGCAAGGCGATTAAGTTGGGTAACGCCAGGGTTTTCCCAGTCACGACGTTGTAAAACGACGGCCAGTAGGATCGCAAGAATTTATTTCTAAATCTATGAGAGATCAACGAAAAGGAAGAGTAGGAAGAGTTAATCCTGGGACATACGTATATTTCTATGATCTGTCTTATATGAAGTCTATACAGCGAATAGATTCAGAATTTCTACATAATTATATATTGTACGCTAATAAGTTTAATCTAACACTCCCCGAAGATTTGTTTATAATCCCTACAAATTTGGATATTCTATGGCGTACAAAGGAATATATAGACTCGTTCGATATTAGTACAGAAACATGGAATAAATTATTATCCAATTATTATATGAAGATGATAGAGTATGCTAAACTTTATGTACTAAGTCCTATTCTCGCTGAGGAGTTGGATAATTTTGAGAGGACGGGAGAATTAACTAGTATTGTACAAGAAGCCATTTTATCTCTAAATTTACGAATTAAGATTTTAAATTTTAAACATAAAGATGATGATACGTATATACACTTTTGTAAAATATTATTCGGTGTCTATAACGGAACAAACGCTACTATATATTATCATAGACCTCTAACGGGATATATGAATATGATTTCAGATACTATATTTGTTCCTGTAGATAATAACTAAAACGGCCGTTTCATTTTGTTTTTTTCTATGCTATAAATGGTGAGCAAGGGCGAGGAGCTGTTCACCGGGGTGGTGCCCATCCTGGTCGAGCTGGACGGCGACGTAAACGGCCACAAGTTCAGCGTGTCCGGCGAGGGCGAGGGCGATGCCACCTACGGCAAGCTGACCCTGAAGTTCATCTGCACCACCGGCAAGCTGCCCGTGCCCTGGCCCACCCTCGTGACCACCCTGACCTACGGCGTGCAGTGCTTCAGCCGCTACCCCGACCACATGAAGCAGCACGACTTCTTCAAGTCCGCCATGCCCGAAGGCTACGTCCAGGAGCGCACCATCTTCTTCAAGGACGACGGCAACTACAAGACCCGCGCCGAGGTGAAGTTCGAGGGCGACACCCTGGTGAACCGCATCGAGCTGAAGGGCATCGACTTCAAGGAGGACGGCAACATCCTGGGGCACAAGCTGGAGTACAACTACAACAGCCACAACGTCTATATCATGGCCGACAAGCAGAAGAACGGCATCAAGGTGAACTTCAAGATCCGCCACAACATCGAGGACGGCAGCGTGCAGCTCGCCGACCACTACCAGCAGAACACCCCCATCGGCGACGGCCCCGTGTTGCTGCCCGACAACCACTACCTGAGCACCCAGTCCGCCCTGAGCAAAGACCCCAACGAGAAGCGCGATCACATGGTCCTGCTGGAGTTCGTGACCGCCGCCGGGATCACTCTCGGCATGGACGAGCTGTACAAGTAAGGTTGAGAGGACGGGAGAATTAACTAGTATTGTACAAGAAGCCATTTTATCTCTAAATTTACGAATTAAGATTTTAAATTTTAAACATAAAGATGATGATACGTATATACACTTTTGTAAAATATTATTCGGTGTCTATAACGGAACAAACGCTACTATATATTATCATAGACCTCTAACGGGATATATGAATATGATTTCAGATACTATATTTGTTCCTGTAGATAATAACTAAAAGGTCAAGCTTGGTACCCAACCTAAAAATTGAAAATAAATACAAAGGTTCTTGAGGGTTGTGTTAAATTGAAAGCGAGAAATAATCATAAATAAGCCCAAACCATGAAGCTACTAATGGTCCTAATGCTAGCAGCACTATCTCAACACTGTTATGCTGGATCTGGATGTCCATTGCTAGAAAACGTAATCTCCAAGACCATCAATCCACAGGTCTCTAAGACCGAGTACAAAGAGCTACTACAAGAGTTCATCGACGACAATGCGACAACAAATGCGATCGACGAGCTAAAAGAGTGCTTCCTAAACCAGACCGACGAAACCCTATCCAACGTCGAAGTATTCATGCAGCTAATCTACGACTCCTCCTTGTGCGACCTATTCGGATCTGGAGCTACCAATTTTAGCCTGCTGAAACAGGCCGGCGACGTAGAAGAAAATCCTGGACCTGCTTTCCTATGGCTACTATCTTGTTGGGCTCTATTGGGAACAACATTCGGACCACTAGAACAGAGATCCCAGCACTGTAAACCAGAAGAAGGACTAGAAGCTAGAGGAGAGGCTCTAGGATTAGTTGGAGCACAAGCTCCAGCGACAGAAGAACAAGAAGCTGCCTCTTCTTCCTCCACGTTGGTCGAAGTAACATTGGGAGAAGTACCAGCTGCAGAATCACCAGATCCTCCACAATCTCCACAAGGTGCTAGTTCTCTACCGACCACAATGAACTATCCGTTATGGTCCCAGTCCTACGAGGACTCTTCTAATCAAGAGGAGGAAGGACCGTCCACCTTTCCAGATCTAGAATCTGAATTTCAAGCTGCGTTGTCTAGAAAGGTAGCTGAACTAGTACACTTCCTACTATTGAAGTACAGAGCGAGAGAGCCAGTAACAAAAGCTGAAATGCTAGGATCTGTAGTAGGAAACTGGCAGTACTTCTTCCCAGTGATCTTCTCCAAAGCTTCTTCCAGTCTACAATTGGTATTCGGAATCGAGCTAATGGAAGTCGACCCAATCGGACACTTGTACATCTTCGCTACCTGTCTAGGACTATCGTACGACGGACTATTGGGAGATAATCAGATCATGCCAAAGGCTGGATTACTAATCATCGTCCTAGCGATTATTGCGAGAGAAGGAGATTGTGCTCCAGAAGAAAAGATCTGGGAAGAGTTGTCCGTCCTAGAGGTATTCGAGGGAAGAGAGGATTCCATTTTGGGAGATCCGAAGAAGCTATTGACCCAGCACTTCGTCCAAGAGAACTACCTAGAGTACAGACAGGTCCCAGGATCTGATCCAGCGTGTTACGAATTTCTATGGGGACCAAGAGCTCTAGTCGAAACCTCCTATGTAAAGGTCCTACACCACATGGTCAAGATCTCTGGTGGTCCACATATTTCTTATCCACCACTACATGAATGGGTCCTAAGAGAGGGAGAAGAAGGTTCTGGAGCCACCAACTTCTCGTTGCTAAAACAGGCTGGAGATGTGGAAGAAAACCCTGGACCGAACCTATTGCTAATCCTAACATTCGTTGCTGCTGCAGTAGCTGGTGCTCCTACATTACCACCAGCTTGGCAACCGTTCCTAAAGGACCATAGAATCTCCACCTTTAAGAACTGGCCATTCCTAGAGGGATGTGCGTGTACACCAGAAAGAATGGCTGAAGCTGGATTCATCCATTGTCCGACCGAAAACGAACCAGATTTGGCTCAGTGTTTCTTCTGCTTCAAAGAACTAGAAGGATGGGAGCCAGATGACGATCCGATCGAAGAACACAAAAAGCACTCCTCTGGATGTGCCTTCCTATCCGTAAAGAAGCAGTTCGAAGAGTTGACCTTGGGAGAGTTCTTGAAGTTGGATAGAGAGAGAGCGAAAAACAAGATCGCGAAAGAGACAAACAACAAGAAGAAAGAGTTCGAGGAAACCGCTGAGAAAGTCAGAAGAGCGATTGAACAACTAGCTGCCATGGATGGAAGTGGAGAAGGAAGAGGATCCCTACTAACCTGTGGAGATGTAGAAGAGAATCCAGGACCAAAATGGGTCACCTTCATCTCGCTACTATTCCTTTTCTCCTCTGCGTACTCCTCTCACTACAGAGAAGTACTACCACTAGCGACCTTCGTCAGAAGATTGGGACCACAGGGTTGGAGACTAGTCCAAAGAGGAGATCCAGCTGCTTTCAGAGCTTTGGTAGCTCAGTGTCTAGTATGTGTACCATGGGATGCTAGACCACCTCCAGCAGCTCCATCTTTCAGACAAGTCTCTTGCCTAAAAGAGCTAGTCGCTAGAGTCCTACAGAGACTATGTGAAAGAGGTGCCAAGAACGTACTAGCGTTTGGATTCGCTCTACTAGACGGTGCTAGAGGTGGACCACCAGAAGCTTTTACAACCTCCGTCAGATCTTACCTACCGAACACAGTAACCGATGCACTAAGAGGTTCTGGTGCTTGGGGATTGCTATTGAGAAGAGTCGGAGATGATGTCCTAGTCCACCTATTGGCTAGATGTGCGTTATTCGTACTAGTAGCACCATCTTGTGCGTACCAAGTTTGTGGACCTCCGTTGTATCAATTGGGAGCTGCTACACAAGCTAGACCTCCACCACATGCATCTGGACCTAGAAGAAGATTAGGTTGCGAAAGAGCGTGGAACCACTCTGTTAGAGAAGCTGGTGTACCACTAGGATTACCAGCTCCAGGTGCAAGAAGAAGAGGAGGATCTGCTTCTAGATCTTTACCACTACCAAAAAGACCGAGAAGAGGTGCTGCACCTGAACCTGAAAGAACACCAGTAGGACAAGGTTCTTGGGCTCATCCTGGTAGAACAAGAGGACCATCTGATAGAGGATTCTGCGTAGTATCTCCAGCAAGACCAGCTGAAGAAGCGACATCTTTAGAGGGTGCTCTATCTGGAACAAGACACTCCCATCCATCTGTAGGTAGACAGCATCATGCAGGACCACCGTCTACATCAAGACCACCAAGACCGTGGGATACACCATGTCCACCAGTATACGCTGAAACGAAGCACTTCCTATACTCCAGTGGAGACAAAGAGCAGCTAAGACCGTCCTTCTTGCTATCCAGTCTAAGACCATCTCTAACTGGTGCTAGAAGACTGGTCGAAACCATCTTCCTAGGATCTAGACCATGGATGCCTGGAACACCAAGAAGATTGCCAAGACTACCACAAAGATACTGGCAGATGAGACCGTTATTCCTAGAGTTGTTGGGAAACCATGCTCAATGTCCGTACGGTGTATTGCTAAAGACACACTGTCCTCTAAGAGCTGCTGTAACTCCAGCAGCAGGTGTTTGTGCTAGAGAAAAACCACAAGGATCTGTAGCTGCTCCTGAAGAAGAAGATACAGATCCGAGAAGATTAGTCCAGCTACTAAGACAGCATTCCTCTCCGTGGCAAGTATACGGATTTGTCAGAGCCTGCCTAAGAAGATTGGTTCCACCAGGATTATGGGGATCGAGACACAACGAGAGAAGATTCCTAAGAAACACGAAGAAGTTCATCTCCCTAGGAAAGCATGCGAAGCTATCCCTACAAGAACTAACGTGGAAGATGTCCGTCAGAGATTGCGCCTGGTTGAGAAGATCTCCAGGTGTAGGATGTGTACCTGCAGCTGAACATAGACTAAGAGAAGAGATCCTAGCGAAGTTCCTACACTGGCTAATGTCCGTCTACGTCGTCGAGTTGCTAAGATCGTTCTTCTACGTAACCGAGACAACCTTCCAAAAGAACAGACTATTCTTCTACAGAAAGTCCGTCTGGTCCAAGCTACAGTCCATCGGAATCAGACAGCACCTAAAGAGAGTCCAGTTGAGAGAACTATCTGAGGCTGAAGTCAGACAACACAGAGAAGCAAGACCTGCCTTGCTAACCTCCAGACTAAGATTCATTCCGAAGCCTGATGGACTTAGACCGATCGTCAACATGGATTATGTAGTCGGAGCGAGAACCTTCAGAAGAGAAAAGAGAGCTGAGAGACTAACCTCGAGAGTAAAGGCACTATTCTCCGTCCTAAATTACGAGAGAGCTAGAAGACCTGGACTTTTGGGAGCTTCCGTACTAGGACTAGATGACATCCATAGAGCTTGGAGAACGTTCGTCTTGAGAGTCAGAGCTCAAGATCCACCACCAGAACTATACTTCGTAAAAGTCGATGTCACTGGTGCCTACGATACCATTCCACAGGATAGACTAACCGAAGTGATCGCATCTATCATCAAACCGCAAAACACCTACTGCGTTAGAAGATACGCAGTAGTACAAAAAGCTGCTCATGGACACGTTAGAAAAGCGTTCAAGTCTCATGTCTCTACCCTAACAGATCTACAACCGTACATGAGACAATTCGTCGCTCATCTACAAGAAACCTCTCCACTAAGAGATGCAGTCGTAATCGAACAGTCCTCGTCTTTGAACGAAGCGTCCTCTGGACTATTCGACGTGTTCTTGAGATTCATGTGTCACCACGCAGTCAGAATCAGAGGAAAGTCTTATGTACAGTGCCAGGGAATACCACAGGGATCTATCCTATCTACCCTACTATGCTCCCTATGCTACGGAGACATGGAAAACAAGCTATTCGCTGGTATAAGAAGAGATGGACTTCTACTTAGACTAGTCGACGACTTCCTATTGGTCACACCTCATCTAACACATGCCAAGACATTCTTGAGAACCCTAGTCAGAGGTGTCCCAGAATACGGATGTGTCGTCAACCTAAGAAAGACGGTCGTAAACTTCCCAGTAGAGGATGAAGCACTAGGTGGAACAGCGTTCGTACAAATGCCAGCTCATGGTCTATTCCCTTGGTGTGGACTACTACTAGATACAAGAACCTTGGAGGTCCAGTCCGACTACTCTTCTTATGCGAGAACTTCCATCAGAGCGAGTCTAACCTTCAACAGAGGATTCAAGGCTGGAAGAAACATGAGAAGAAAGTTGTTCGGAGTCCTAAGATTGAAGTGCCACTCCTTGTTCCTAGACCTACAAGTCAACTCCCTACAGACCGTGTGTACCAACATCTATAAGATCTTGCTATTGCAAGCGTACAGATTCCACGCGTGTGTACTACAACTACCCTTTCATCAGCAAGTTTGGAAGAATCCGACATTCTTCCTAAGAGTGATCTCCGACACTGCGAGTCTATGCTACTCTATCCTAAAAGCGAAGAACGCTGGAATGTCTTTGGGAGCAAAAGGTGCAGCTGGACCATTACCATCTGAAGCAGTACAATGGCTATGTCACCAGGCCTTCTTGCTAAAGCTAACCAGACACAGAGTCACCTACGTACCACTACTAGGATCTTTGAGAACAGCACAGACCCAGCTATCTAGAAAGCTACCTGGAACAACACTAACAGCTCTAGAAGCTGCTGCTAATCCAGCATTACCGTCTGATTTCAAGACGATCCTAGATTAAGGATCCTAGTTAATTAAAATCAAACTCTAATGACCACATCTTTTTTTAGAGATGAAAAATTTTCCACATCTCCTTTTGTAGACACGACTAAACATTTTGCAGAAAAAAGTTTATTAGTGTTTAGATAATCGTATACTTCATCAGTGTAGATAGTAAATGTGAACAGATAAAAGGTATTCTTGCTCAATAGATTGGTAAATTCCATAGAATATATTAATCCTTTCTTCTTGAGATCCCACATCATTTCAACCAGAGACGTTTTATCCAATGATTTACCTCGTACTATACCACATACAAAACTAGATTTTGCAGTGACGTCGTATCTGGTATTCCTACCAAACAAAATTTTACTTTTAGTTCTTTTAGAAAATTCTAAGGTAGAATCTCTATTTGCCAATATGTCATCTATGGAATTACCACTAGCAAAAAATGATAGAAATATATATTGATACATCGCAGCTGGTTTTGATCTACTATACTTTAAAAACGAATCAGATTCCATAATTGCCTGTATATCATCAGCTGAAAAACTATGTTTTACACGTATTCCTTCGGCATTTCTTTTTAATGATATATCTTGTTTAGACAATGATAAAGTTATCATGTCCATGAGAGACGCGTCTCCGTATCGTATAAATATTTCATTAGATGTTAGACGCTTCATTAGGGGTATACTTCTATAAGGTTTCTTAATCAGTCCATCATTGGTTGCGTCAAGAACTACTATCGGATGTTGTTAAGCTTGGCGTAATCATGGTCATAGCTGTTTCCTGTGTGAAATTGTTATCCGCTCACAATTCCACACAACATACGAGCCGGAAGCATAAAGTGTAAAGCCTGGGGTGCCTAATGAGTGAGCTAACTCACATTAATTGCGTTGCGCTCACTGCCCGCTTTCCAGTCGGGAAACCTGTCGTGCCAGCTGCATTAATGAATCGGCCAACGCGCGGGGAGAGGCGGTTTGCGTATTGGGCGCTCTTCCGCTTCCTCGCTCACTGACTCGCTGCGCTCGGTCGTTCGGCTGCGGCGAGCGGTATCAGCTCACTCAAAGGCGGTAATACGGTTATCCACAGAATCAGGGGATAACGCAGGAAAGAACATGTGAGCAAAAGGCCAGCAAAAGGCCAGGAACCGTAAAAAGGCCGCGTTGCTGGCGTTTTTCCATAGGCTCCGCCCCCCTGACGAGCATCACAAAAATCGACGCTCAAGTCAGAGGTGGCGAAACCCGACAGGACTATAAAGATACCAGGCGTTTCCCCCTGGAAGCTCCCTCGTGCGCTCTCCTGTTCCGACCCTGCCGCTTACCGGATACCTGTCCGCCTTTCTCCCTTCGGGAAGCGTGGCGCTTTCTCATAGCTCACGCTGTAGGTATCTCAGTTCGGTGTAGGTCGTTCGCTCCAAGCTGGGCTGTGTGCACGAACCCCCCGTTCAGCCCGACCGCTGCGCCTTATCCGGTAACTATCGTCTTGAGTCCAACCCGGTAAGACACGACTTATCGCCACTGGCAGCAGCCACTGGTAACAGGATTAGCAGAGCGAGGTATGTAGGCGGTGCTACAGAGTTCTTGAAGTGGTGGCCTAACTACGGCTACACTAGAAGAACAGTATTTGGTATCTGCGCTCTGCTGAAGCCAGTTACCTTCGGAAAAAGAGTTGGTAGCTCTTGATCCGGCAAACAAACCACCGCTGGTAGCGGTGGTTTTTTTGTTTGCAAGCAGCAGATTACGCGCAGAAAAAAAGGATCTCAAGAAGATCCTTTGATCTTTTCTACGGGGTCTGACGCTCAGTGGAACGAAAACTCACGTTAAGGGATTTTGGTCATGAGATTATCAAAAAGGATCTTCACCTAGATCCTTTTAAATTAAAAATGAAGTTTTAAATCAATCTAAAGTATATATGAGTAAACTTGGTCTGACAGTTACCAATGCTTAATCAGTGAGGCACCTATCTCAGCGATCTGTCTATTTCGTTCATCCATAGTTGCCTGACTCCCCGTCGTGTAGATAACTACGATACGGGAGGGCTTACCATCTGGCCCCAGTGCTGCAATGATACCGCGAGACCCACGCTCACCGGCTCCAGATTTATCAGCAATAAACCAGCCAGCCGGAAGGGCCGAGCGCAGAAGTGGTCCTGCAACTTTATCCGCCTCCATCCAGTCTATTAATTGTTGCCGGGAAGCTAGAGTAAGTAGTTCGCCAGTTAATAGTTTGCGCAACGTTGTTGCCATTGCTACAGGCATCGTGGTGTCACGCTCGTCGTTTGGTATGGCTTCATTCAGCTCCGGTTCCCAACGATCAAGGCGAGTTACATGATCCCCCATGTTGTGCAAAAAAGCGGTTAGCTCCTTCGGTCCTCCGATCGTTGTCAGAAGTAAGTTGGCCGCAGTGTTATCACTCATGGTTATGGCAGCACTGCATAATTCTCTTACTGTCATGCCATCCGTAAGATGCTTTTCTGTGACTGGTGAGTACTCAACCAAGTCATTCTGAGAATAGTGTATGCGGCGACCGAGTTGCTCTTGCCCGGCGTCAATACGGGATAATACCGCGCCACATAGCAGAACTTTAAAAGTGCTCATCATTGGAAAACGTTCTTCGGGGCGAAAACTCTCAAGGATCTTACCGCTGTTGAGATCCAGTTCGATGTAACCCACTCGTGCACCCAACTGATCTTCAGCATCTTTTACTTTCACCAGCGTTTCTGGGTGAGCAAAAACAGGAAGGCAAAATGCCGCAAAAAAGGGAATAAGGGCGACACGGAAATGTTGAATACTCATACTCTTCCTTTTTCAATATTATTGAAGCATTTATCAGGGTTATTGTCTCATGAGCGGATACATATTTGAATGTATTTAGAAAAATAAACAAATAGGGGTTCCGCGCACATTTCCCCGAAAAGTGCCACCTGACGTCTAAGAAACCATTATTATCATGACATTAACCTATAAAAATAGGCGTATCACGAGGCCCTTTCGTC

SEQ ID NO: 8 - protein, 1777 aa, Artificial Sequence; identical to SEQ ID NO: 6
  Synthetic Sequence Antigen protein sequence encoded by mvaBC.2 (pMVa-mH5_Mamma_Magea3_Survivin_Tert)

(19) United States
(12) Patent Application Publication
(10) Pub. No.: US 2023/0233656 A1
(43) Pub. Date: Jul. 27, 2023

(54) Breast Cancer Vaccine

(71) Applicant: National Breast Cancer Coalition, Washington, DC (US)

(72) Inventor: Keith L. Knutson, Jacksonville, FL (US)

(73) Assignee: National Breast Cancer Coalition, Washington, DC (US)

(21) Appl. No.: 18/012,432

(22) PCT Filed: Jun. 25, 2021
(86) PCT No.: PCT/US2021/039046

§ 371 Date: Dec. 22, 2022

Related U.S. Application Data
(60) Provisional application No. 63/044,675, filed on Jun. 26, 2020.

(51) Int. Cl. A61K 39/00; C12N 15/86

(52) U.S. Cl. CPC A61K 39/001106; A61K 39/00115; A61K 39/00117; A61K 39/001186; C12N 15/86; A61K 2039/53; A61K 2039/812; C12N 2710/24143

(57) ABSTRACT

The invention relates to vaccines for breast cancer therapy. The invention also relates to methods of preventing and treating breast cancer using a breast cancer vaccine.

### BACKGROUND OF THE INVENTION

As it is associated with approximately 459,000 deaths each year, breast cancer engenders an extremely high burden globally. According to a Swedish study, costs for any medical condition, fall into one of three categories: direct costs (costs directly linked to treatment, detection, prevention or care); indirect costs (lost productivity and premature mortality); and intangible costs (reduced quality of life). As such, the average annual total cost of breast cancer care in the USA might be estimated as approximately $170 billion per year; about 1% of the United States gross domestic product. While the incidence is roughly stable in industrialized countries, it is rising sharply in developing countries, attributable to changes in reproductive factors, lifestyles, and life expectancies. As a result, economic costs will escalate sharply, particularly as we continue to implement newer early detection and novel expensive therapeutic strategies; an extraordinary burden is therefore placed on limited global resources. In response, we must reposition ourselves and develop a serious interest in advancing promising, affordable and safe strategies for the primary prevention.

To both curb cancer-associated morbidity and mortality, and alleviate the economic burden of treatment, strategies are being developed for cancer prevention. Chemopreventive treatments (involving chronic administration of a synthetic, natural, or biological agent) have been successful for subtypes of breast cancer (Advani and Moreno-Aspitia (2014) Breast Cancer. 6: 59-71). In women at high risk for breast cancer for example, treatment with selective estrogen receptor modulators has significantly reduced the risk of onset (Advani and Moreno-Aspitia, 2014). However, such approaches, involving long-term administration of preventive agents, have been associated with serious side effects. These include an increased risk of developing other types of cancers and cardiovascular disease (Advani and Moreno-Aspitia, 2014). In this context, there is significant interest in creating alternative approaches, such as breast cancer vaccines. To prevent infectious disease and (more recently) cancer (e.g. cervical cancer), vaccines have both outstanding efficacy and safety profiles. As such, a vaccine strategy can circumvent challenges associated with traditional chemoprevention. Cancer prevention through vaccination has been successful in numerous animal models (Disis et al. (2013) Cancer Prev. Res. 6: 1273-82; Lollini et al. (2006) Nat. Rev. Cancer, 6: 204-16; Nava-Parada et al. (2007) Cancer Res. 67: 1326-34). Nevertheless, upon translation to clinic, the majority of vaccine technologies are applied as therapies, primarily in patients with advanced disease. They are deployed for disease elimination rather than deterrence. Therapeutic cancer vaccinations, in both animal models and clinical trials, have experienced some success. One example is the first FDA-approved therapeutic vaccine for prostate cancer, sipuleucel-T.

Nevertheless, there are several challenges associated with therapeutic vaccination aimed at disease elimination: (1) Tumor growth kinetics may outstrip the immune system's potential for removal; (2) the tumor has already orchestrated strategies to suppress the immune response; and (3) the tumors may have developed mechanisms to reduce their susceptibility to immune-mediated destruction. Evasion strategies include reduced antigen expression or reduced HLA expression. They also include suppression of immune cytotoxic mechanisms. When addressing a disease associated with these issues, a vaccine-induced immune response, that successfully eradicates the cancer and produces a cure, is an unlikely outcome.

A breast cancer preventive vaccine, administered before the appearance of tumor, adventitiously bypasses many of the challenges associated with therapeutic use. As such, preventive vaccination can both build upon and enhance the success of the therapeutic approach. To achieve this, a preventive vaccine must target antigens which are widely expressed across the various subtypes of breast cancer. The antigens must also be disease specific. One approach to do this has garnered significant enthusiasm: leveraging normal self-proteins. In various types of cancers and cancer precursors, these self-proteins are overexpressed. This aberrant overexpression of self-proteins leads to presentation of subdominant, tumor-specific epitopes: prime targets for a preventive vaccine (Disis et al., 2002; Moudgil (1999) Immunol. Lett. 68: 251-6; Reynolds et al. (1996) J. Exp. Med. 184: 1857-70).

### SUMMARY OF THE INVENTION

The invention relates to an immunogenic composition comprising a vector encoding at least one epitope from at least three antigens encoded by a gene selected from the group consisting of MUC1, HER2, hTERT, Survivin, MAGEA3 and Mammaglobin A, and at least one pharmaceutically acceptable excipient. In some embodiments, the vector encodes at least four antigens encoded by a gene selected from the group consisting of MUC1, HER2, hTERT, Survivin, MAGEA3 and Mammaglobin A. In some embodiments, the vector encodes at least five antigens encoded by a gene selected from the group consisting of MUC1, HER2, hTERT, Survivin, MAGEA3 and Mammaglobin A. In some embodiments, the vector encodes at least six antigens encoded by a gene selected from the group consisting of MUC1, HER2, hTERT, Survivin, MAGEA3 and Mammaglobin A.

In some embodiments, the at least one epitope encoded by the MUC1 gene comprises the full length protein encoded by the MUC1 gene. In some embodiments, the at least one epitope encoded by the HER2 gene comprises amino acids 1 to 652 of the protein encoded by the HER2 gene. In some embodiments, the at least one epitope encoded by the HER2 gene comprises amino acids 676 to 1254 of the protein encoded by the HER2 gene. In some embodiments, the at least one epitope encoded by the hTERT gene comprises amino acids 15 to 1132 of the protein encoded by the hTERT gene. In some embodiments, the at least one epitope encoded by the MAGEA3 gene comprises the full length protein encoded by the MAGEA3 gene. In some embodiments, the at least one epitope encoded by the Survivin gene comprises the full length protein encoded by the Survivin gene. In some embodiments, each epitope is separated by a cleavable spacer sequence. In some embodiments, the vector is a DNA vector. In some embodiments, the vector is a Modified Vaccinia Ankara (MVA) virus.

The invention also relates to a method of inducing an antibody-mediated or T cell mediated immune response in a human subject against one or more antigens selected from the group consisting of MUC1, HER2, hTERT, Survivin, MAGEA3 and Mammaglobin A comprising administering to the human subject the immunogenic composition described herein. In some embodiments, the method comprises administering the immunogenic composition to the human subject. In some embodiments, the immunogenic composition containing the vaccinia vector is administered to the human subject after administration of the immunogenic composition containing the plasmid vector. In some embodiments, the vaccinia vector is administered to the human subject at least 15 days after administration of the plasmid vector. In some embodiments, it is administered to the human subject at least 30 days after. In some embodiments, the human subject is not suffering from breast cancer. In some embodiments, the human subject is suffering from primary breast cancer. In some embodiments, the human subject is suffering from metastatic breast cancer. In some embodiments, the human subject is suffering from Stage 0, I, II, III or IV breast cancer. In some embodiments, the human subject is not receiving chemotherapy.

### BRIEF DESCRIPTION OF DRAWINGS

FIG. 1: For Artemis 1.P1 and Artemis 1.V1, the plasmids contain HER2 extracellular domain (ECD), MUC1, and HER2 intracellular domain (ICD). For Artemis 1.P2 and Artemis 1.V2, the plasmids contain mammoglobin A, MAGEA3, survivin, and hTERT.

FIG. 2: pUMVC4a plasmid diagram. For plasmid vaccination, pUMVC4a plasmid, a derivative of pNGVL-3 vector, is used in clinical trials and has several unique features amenable to vaccination including an immuno-stimulatory sequence (ISS) and an intron for enhanced transcript stability.

FIG. 3: pMVApIleGFP-mH5 transfer plasmid diagram. This plasmid allows for transient selection under EGFP, recombinant expression under the mH5 early/late promoter, and insertion into the I8RL/G1L site of the MVA genome.

FIG. 4: Quality control of recombinant MVA. PCR was performed using DNA from mvaBC.1 and mvaBC.2 positive controls. Amplicons were of correct size and 100% sequence identity (by sequencing) to their originating plasmid.

FIG. 5: Western blot analysis of cellular and secreted (supernatant) fractions from pBC.1 and pBC.2 plasmid transfected HEK293 cells.

FIG. 6: Western blot analysis of cellular and secreted (supernatant) fractions from mvaBC.1 and mvaBC.2 infected DF-1 cells.

FIG. 7: Vector map for pBC.1 (pUMVC4a-HER2ECD_Mucin1_HER2ICD) plasmid (see SEQ ID NO: 1 & 2 for coding and amino acid sequences).

FIG. 8: Vector map for mvaBC.1 (pMVa-mH5_HER2ECD_Mucin1_HER2ICD) vector (see SEQ ID NO: 3 & 4 for coding and amino acid sequences).

FIG. 9: Vector map for pUMVC4a-Mamma_Magea3_Survivin_Tert plasmid (see SEQ ID NO: 5 & 6 for coding and amino acid sequences).

FIG. 10: Vector map for pMVa-mH5_Mamma_Magea3_Survivin_Tert vector (see SEQ ID NO: 7 & 8 for coding and amino acid sequences).

### DETAILED DESCRIPTION OF THE INVENTION

In describing and claiming the invention, the following terminology will be used in accordance with the definitions set forth below.

An “immunogenic composition” or “vaccine” as used herein refers to any one or more compounds or agents or immunogens capable of priming, potentiating, activating, eliciting, stimulating, augmenting, boosting, amplifying, or enhancing an adaptive (specific) immune response, which may be cellular (T cell) or humoral (B cell), or a combination thereof. Preferably, the adaptive immune response is protective. A representative example of an immunogen is from a MUC1, HER2, hTERT, Survivin, MAGEA3 and Mammaglobin A antigen. In the present description, any concentration range, percentage range, ratio range, or integer range is understood to include the value of any integer within the recited range and, when appropriate, fractions thereof (such as one tenth and one hundredth of an integer, etc.), unless otherwise indicated.

The term “Immunogenicity” means the ability of a MUC1, HER2, hTERT, Survivin, MAGEA3 and Mammaglobin A antigen to evoke an immune response directed to any of MUC1, HER2, hTERT, Survivin, MAGEA3 and Mammaglobin A proteins. Whether a preparation is immunogenic can be tested by, for instance, a DTH-assay or an in vivo assay in an experimental animal model.

As used herein, the term “vector” may mean a nucleic acid sequence containing an origin of replication. A vector may be a plasmid, virus, bacteriophage, bacterial artificial chromosome or yeast artificial chromosome. A vector may be a DNA or RNA vector. A vector may be either a self-replicating extrachromosomal vector or a vector which integrates into a host genome.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans.

As used herein, the term “treating” includes prophylaxis of the specific disorder or condition, or alleviation of the symptoms associated with a specific disorder or condition and/or preventing or eliminating said symptoms.

As used herein an “effective” amount or a “therapeutically effective amount” of a pharmaceutical refers to a nontoxic but sufficient amount of the pharmaceutical to provide the desired effect. For example, one desired effect would be the prevention or treatment of breast cancer. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

As used herein a “linker” is a bond, molecule or group of molecules that binds two separate entities to one another. Linkers may provide for optimal spacing of the two entities or may further supply a labile linkage that allows the two entities to be separated from each other. Labile linkages include enzyme-cleavable groups.

As used herein the term “patient” without further designation is intended to encompass humans.

As used herein, “about” or “comprising essentially of” means±10%. As used herein, the use of an indefinite article, such as “a” or “an,” should be understood to refer to the singular and the plural of a noun or noun phrase (i.e. meaning “one or more” or “at least one” of the enumerated elements or components). The use of the alternative (e.g. “or”) should be understood to mean either one, both or any combination thereof of the alternatives. When ranges are used herein to describe, for example, physical or chemical properties such as molecular weight or chemical formulae, all combinations and subcombinations of ranges and specific embodiments therein are intended to be included. Use of the term “about” when referring to a number or a numerical range means that the number or numerical range referred to is an approximation within experimental variability (or within statistical experimental error), and thus the number or numerical range may vary. The variation is typically from 0% to 15%, preferably from 0% to 10%, more preferably from 0% to 5% of the stated number or numerical range. The term “comprising” (and related terms such as “comprise” or “comprises” or “having” or “including”) includes those embodiments such as, for example, an embodiment of any composition of matter, method or process that “consist of” or “consist essentially of” the described features.

### Immunogenic Compositions

In accordance with one embodiment a multivalent antigenic (i.e. immunogenic) composition is provided for inducing an immune response in a patient. In one embodiment, the multivalent antigenic composition is administered prophylactically to prevent breast cancer. In one illustrative aspect, the composition is administered to non-lactating women at risk for developing breast cancer. Alternatively, in an embodiment the composition is administered, optionally in conjunction with other know anti-cancer therapies, to treat breast cancer. In accordance with some embodiments the multivalent antigenic composition is a vector encoding three or more, four or more, five or more, or six proteins or antigenic fragments thereof, of proteins encoded by any of the genes selected from MUC1, HER2, hTERT, Survivin, MAGEA3 and Mammaglobin A.

Immunogenic compositions or vaccines as described herein useful for treating and/or preventing breast cancer comprise one or more vectors, two or more vectors encoding genes selected from MUC1, HER2, hTERT, Survivin, MAGEA3 and Mammaglobin A, fragments and variants thereof. In certain embodiments, the vectors may encode any portion of these proteins that has an epitope capable of eliciting a protective immune response (e.g. eliciting production of a neutralizing antibody and/or stimulating a cell-mediated immune response). Plasmids may have these antigens or fragments thereof as described herein arranged, combined, or fused in a linear form, and each immunogen may or may not be reiterated, wherein the reiteration may occur once or multiple times, and may be located at the N-terminus, C-terminus, or internal to a linear sequence of the antigen. In addition, a plurality of different antigen polypeptides can be selected or combined into a one or more vectors to provide a multivalent vaccine for use in eliciting a protective immune response without a harmful or otherwise unwanted associated immune responses or side effects.

In an embodiment, a multivalent human breast cancer vaccine comprises a vector encoding six immunogenic polypeptides or fragments thereof. In an embodiment, one of the immunogenic polypeptides comprises human Mucin-1 (MUC1). The MUC1 gene (see SEQ ID NO: 1 & 3 for exemplary embodiments) encodes a large, transmembrane mucin glycoprotein (see SEQ ID NO: 2 & 4 for exemplary embodiments) expressed at the apical surface of a variety of epithelial cells. Like other mucins, Mucin-1 is involved in lubrication and hydration of the epithelial cell surface as well as protection from microorganisms and degradative enzymes. The molecular weight is 125-225 kilodaltons for the unglysolated form and due to allelic variation in the extracellular domain repeat motifs. Typically, the protein is heavily glycosylated yielding proteins with molecular weights in the range of 225 to 500 kilodaltons. MUC1 is overexpressed and aberrantly glycosylated in many human cancers including breast cancer.

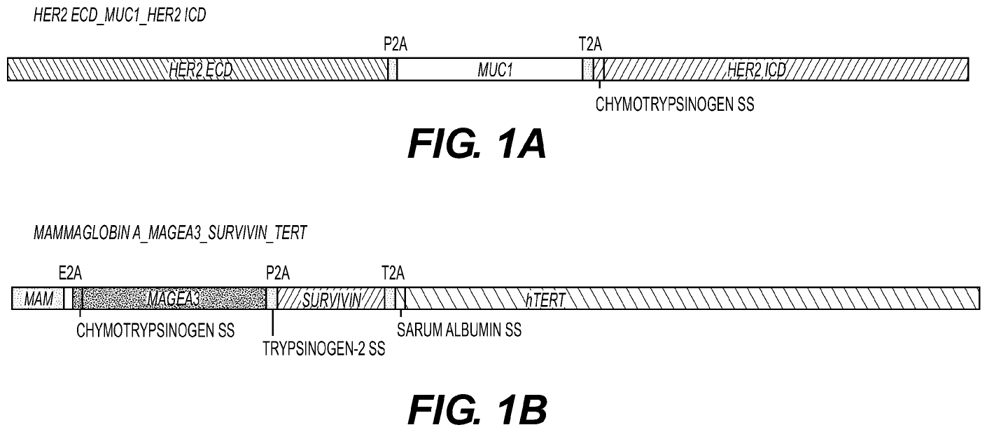

In an embodiment, a multivalent human breast cancer vaccine comprises a vector encoding six immunogenic polypeptides or fragments thereof. In an embodiment, one of the immunogenic polypeptides comprise human epidermal growth factor receptor 2 (HER2) or antigenic fragments thereof (see SEQ ID NO: 2 & 4 for exemplary embodiments). In some embodiments, the vector encodes one or more antigenic fragments of HER2 (see SEQ ID NO: 1 & 3 for exemplary embodiments). In an embodiment, the vector encodes to distinct antigenic fragments of HER2. In an embodiment, the vector encodes one antigenic fragment comprising or consisting of the amino acids in the extracellular domain of HER2. In an embodiment, the vector encodes one antigenic fragment comprising or consisting of amino acids 1 to 652 of HER2 (see FIG. 1). In an embodiment, the vector encodes one antigenic fragment comprising or consisting of the amino acids in the intracellular domain of HER2. In an embodiment, the vector encodes one antigenic fragment comprising or consisting of amino acids 676-1254 of HER2 (see FIG. 1). In an embodiment, the vector encodes two separate and distinct antigenic fragments of HER2 separated by a gene encoding another antigen disclosed herein or a fragment thereof. In some embodiments, the another antigen is MUC1 or a fragment thereof. The HER2 proto-oncogene encodes a non-mutated, 185 kD transmembrane glycoprotein receptor with extensive homology to the epidermal growth factor receptor. The HER2 protein consists of a large cysteine-rich extracellular domain (ECD) which probably functions in ligand binding, a short transmembrane domain, and a small cytoplasmic domain with tyrosine kinase activity (ICD). In normal adult tissue cells, the HER2 gene is present as a single copy. Amplification of the gene or overexpression by post-transcriptional mechanisms leads to overexpression of the associated protein and, thus, plays a role in malignant transformation by contributing to the uncontrolled growth of cancer cells. HER2 overexpression has been described in a variety of different tumor types including breast, ovarian, renal cell, prostate, pancreas, colon, non-small cell lung, gastric, salivary, bladder and oral squamous cell carcinomas. HER2 overexpression regarded as a poor prognostic factor in patients with both node-positive and negative breast cancer. In addition, HER2 overexpression appears to be a predictive factor for resistance to some chemotherapeutic agents. In the neoadjuvant, adjuvant and metastatic settings, HER2 has been successfully targeted with various targeted agents including trastuzumab, pertuzumab, and lapatinib.

In an embodiment, a multivalent human breast cancer vaccine comprises a vector encoding six immunogenic polypeptides or fragments thereof. In an embodiment, one of the immunogenic polypeptides comprises human telomerase reverse transcriptase (hTERT). In an embodiment, the vector (see SEQ ID NO: 5 & 7 for exemplary embodiments) encodes an antigenic fragment of hTERT comprising or consisting of amino acids 15 to 1132 of the hTERT protein (see SEQ ID NO: 6 & 8 for exemplary embodiments). hTERT is the main protein component of telomerase, an enzyme that maintains telomeres on chromosomes and protects them from abnormally sticking together or breaking down (degrading). hTERT is a large protein with a molecular weight about 126 kilodaltons. The protein is largely absent from normal non-dividing cells but remains highly expressed in >90% of cancer cells, including breast cancers.

In an embodiment, a multivalent human breast cancer vaccine comprises a vector encoding six immunogenic polypeptides or fragments thereof. In an embodiment, one of the immunogenic polypeptides comprises human surviving (see SEQ ID NO: 6 & 8 for exemplary embodiments). Survivin is a 16 kilodalton anti-apoptotic protein that, in humans, is encoded by the BIRC5 gene (see SEQ ID NO: 5 & 7 for exemplary embodiments). It also called baculoviral inhibitor of apoptosis repeat-containing 5 or BIRC5. It has been shown that survivin inhibits both Bax and Fas-induced apoptotic pathways. Expression of the protein is found ubiquitously during embryonic and fetal development but not in normal differentiated adult cells. The gene is expressed in greater than 90% breast cancers.

In an embodiment, a multivalent human breast cancer vaccine comprises a vector encoding six immunogenic polypeptides or fragments thereof. In an embodiment, one of the immunogenic polypeptides comprises human melanoma associated antigen A3 (MAGEA3). MAGEA3 in a 34 kilodalton intracellular protein (see SEQ ID NO: 8 & 10 for exemplary embodiments) encoded by the MAGE-A3 gene (see SEQ ID NO: 7 & 9 for exemplary embodiments). Expression of MAGEA3 is limited to placental trophoblast cells and germ cells of the testes. Many cancers overexpress MAGE3, including over half of all breast cancers. The normal physiologic function of MAGEA3 is not known.

In an embodiment, a multivalent human breast cancer vaccine comprises a vector encoding six immunogenic polypeptides or fragments thereof. In an embodiment, one of the immunogenic polypeptides comprises human mammaglobin A (see SEQ ID NO: 6 & 8 for exemplary embodiments). Mammaglobin A, also known as secretoglobin family 2A member 2, is a protein that in humans is encoded by the SCGB2A2 gene (see SEQ ID NO: 5 & 7 for exemplary embodiments). Mammaglobin A is a 10 kilodalton secretory protein 26 that is expressed exclusively in 40 to 80% of primary and metastatic breast cancers. Expression of Mammaglobin A appears to be very limited in normal healthy tissues and it is estimated that expression in breast cancer is ten-fold higher than normal cells. The normal physiologic function, however, of Mammaglobin A is not known.

In accordance with an embodiment of the invention, a multivalent antigenic composition is a vector encoding three, four, five or six of the above referenced antigenic polypeptides or fragments thereof. In some embodiments. In an embodiment, the vector encodes a spacer sequence between each of gene encoding each antigen or antigenic fragment thereof. In some embodiments, the spacer sequence is a cleavable amino acid sequence. In some embodiments, the vector encodes an in frame cleavable amino acid sequence consisting of GSG spacer to allow for cleavage and co-expression.

In an embodiment, the multivalent antigenic composition comprises a vector encoding any of the MUC1, HER2, hTERT, Survivin, MAGEA3 and Mammaglobin A polypeptide which differ by a single amino acid modification relative to the normal amino acid sequence disclosed herein, wherein the amino acid modification is a substitution, deletion or insertion of an amino acid or a post translational modification of an amino acid. In one embodiment the single amino acid modification is a conservative amino acid substitution.

In an embodiment, the multivalent antigenic composition comprises a vector encoding any of the MUC1, HER2, hTERT, Survivin, MAGEA3 and Mammaglobin A antigenic fragments comprising a 15 amino acid fragment of any of MUC1, HER2, hTERT, Survivin, MAGEA3 and Mammaglobin A proteins. In an embodiment, the multivalent antigenic composition comprises a vector encoding any of the MUC1, HER2, hTERT, Survivin, MAGEA3 and Mammaglobin A antigenic fragments comprising a 20 amino acid fragment of any of MUC1, HER2, hTERT, Survivin, MAGEA3 and Mammaglobin A proteins. In an embodiment, the multivalent antigenic composition comprises a vector encoding any of the MUC1, HER2, hTERT, Survivin, MAGEA3 and Mammaglobin A proteins, wherein the amino acid sequence has been modified by substitution, deletion or insertion of an amino acid such that the resulting amino acid sequences has at least 95, 96, 97, 98 or 99% sequence identity with the wild-type amino acid sequence.

In an embodiment the polypeptides of the antigenic compositions linked to one another through a linking moiety. In one embodiment the polypeptides are linked in a head to tail fashion (i.e., the amino terminus of one polypeptide is linked to the carboxy terminus of a second polypeptide). In a further embodiment the polypeptides are linked by an amino acid linker, and in one embodiment the linker is a dipeptide or tripeptide. Typically the linking amino acids are selected from glycine and alanine and in one embodiment the polypeptides are linked with a G-G or A-A-A linker. It is appreciated that the antigenic proteins upon expression following administration are processed in vivo by proteases to smaller peptide fragments, which are able to bind to MHC class I and/or MHC class II molecules on antigen presenting cells. Subsequently, T-cell receptors recognize and bind to the MHC molecule to which the peptide is bound, forming the primary signal that initiates an immune response.

In one embodiment, the vaccine further comprises an adjuvant and a pharmaceutically acceptable carrier. As used herein, the term “‘adjuvant” refers to an agent that stimulates the immune system and increases the response to a vaccine. Vaccine adjuvants are well-known to those of skill in the art. Illustratively, GPI-0100 is a suitable adjuvant for a vaccine. As used herein, the term “carrier” refers to an ingredient other than the active component(s) in a formulation. The choice of carrier will to a large extent depend on factors such as the particular mode of administration or application, the effect of the carrier on solubility and stability, and the nature of the dosage form. Pharmaceutically acceptable carriers for polypeptide antigens are well known in the art. In one embodiment, the vaccine is administered prophylactically to prevent breast cancer. In one illustrative aspect, the vaccine is administered to non-lactating women at risk for developing breast cancer.

In an embodiment, the immunogenic composition is administered to inhibit tumor cell expansion. The immunogenic composition may be administered prior to or after the detection of breast tumor cells in a human patient. Inhibition of tumor cell expansion is understood to refer to preventing, stopping, slowing the growth, or killing of tumor cells. In an illustrative aspect, T cells of the human immune system are activated after administration of a vector based immunogenic composition, and subsequent expression of human MUC1, HER2, hTERT, Survivin, MAGEA3 and Mammaglobin A proteins. The activated T cells may be CD4+ and/or CD8+ cells. In an embodiment, after administration of the immunogenic composition, and subsequent antigenic protein expression, a proinflammatory response is induced by subsequent encounter of immune cells with MUC1, HER2, hTERT, Survivin, MAGEA3 and Mammaglobin A proteins. The proinflammatory immune response comprises production of proinflammatory cytokines and/or chemokines, for example, interferon gamma (IFNγ) and/or interleukin 2 (IL-2). Proinflammatory cytokines and chemokines are well known in the art.

It is to be appreciated that when the breast cancer vaccine is administered to patients whose breast tissue is not actively producing human MUC1, HER2, hTERT, Survivin, MAGEA3 and Mammaglobin A proteins in appreciable quantities, immunization with these antigenic proteins does not elicit a substantial inflammatory immune response (i.e. that is capable of causing breast tissue failure) in breast tissue. Subsequent encounters with these antigenic proteins, such as that expressed by cells of a developing tumor elicits a recall response by the immune system. The recall response includes, but is not limited to, an increase in the production of proinflammatory cytokines such as IFNγ and IL-2, which promote a robust immune system attack against the MUC1, HER2, hTERT, Survivin, MAGEA3 and Mammaglobin A protein expressing cells.

In one embodiment, a method of immunizing a human patient against MUC1, HER2, hTERT, Survivin, MAGEA3 and Mammaglobin A proteins is disclosed. The method comprises the step of administering to the patient an immunogenic composition comprising a vector encoding three or more, four or more, five or more, or six polypeptides selected from human MUC1, HER2, hTERT, Survivin, MAGEA3 and Mammaglobin A proteins. In one aspect, the immunogenic composition comprises a vector encoding all six antigenic polypeptides or one or more antigenic fragments thereof.

In an embodiment, the immunogenic composition is a vaccine for preventing or treating breast cancer. The vaccine comprises an immunogenic polypeptide comprises a vector encoding MUC1, HER2, hTERT, Survivin, MAGEA3 and Mammaglobin A proteins or one or more antigenic fragments thereof.

In an embodiment, a method of treating breast cancer in a human patient is disclosed. The method comprises the step of administering to the patient an immunogenic composition comprising a vector encoding three or more, four or more, five or more, or six polypeptides selected from human MUC1, HER2, hTERT, Survivin, MAGEA3 and Mammaglobin A proteins, and a pharmaceutically acceptable carrier, in an amount effective to induce a breast tissue specific inflammatory response in the human patient.

According to various embodiments for treatment or prevention of breast cancer, one or more booster injections of the immunogenic are administered. T cells recognize discrete peptides of protein antigens presented in the context of antigen presenting molecules that are typically expressed on macrophages and dendritic cells of the immune system. Peptide recognition typically occurs following phagocytic processing of the antigen by antigen-presenting cells and loading of small peptide fragments onto Major Histocompatibility Complex (MHC) class I and/or class II molecules. After CD4+ T cells recognize peptides presented on MHC class II molecules, they proliferate rapidly and become effector T cells that may activate other immune effector cells.

The multivalent immunogenic composition may be administered serially or in combination with other therapeutics used in the treatment of breast cancer. These therapeutics include IFN-alpha, IFN-beta, interleukin-1, interleukin-2, tumor necrosis factor, macrophage colony stimulating factor, macrophage activation factor, lympho-toxin, fibroblast growth factor. Alternatively, the multivalent vaccine may be administered serially or in combination with conventional chemotherapeutic agents such as 5-fluoro uracil; paclitaxel; etoposide; carboplatin; cisplatin; topotecan, methatroxate, and/or radiotherapy. Such combination therapies may advantageously utilize less than conventional dosages of those agents, or involve less radical regimens, thus avoiding any potential toxicity or risks associated with those therapies.

In accordance with an embodiment of the invention, the antigenic polypeptides may be expressed recombinantly following administration of the vector encoding these antigenic polypeptides or fragments thereof including expressing several of the polypeptides linked together as fusion peptides. In one embodiment the multivalent vaccine can be administered in any pharmaceutically acceptable form, intratumorally, peritumorally, interlesionally, intravenously, intramuscularly or subcutaneously.

The antigenic polypeptide fragments of the invention may be of from as small as at least 50, 100, 150, 200, 250, 300, 350, 400, 450, 500, 550, 600 or more amino acids in length. For example, an antigenic polypeptide fragment of the invention may be the minimal length require to produce an epitope which is recognized by one or more antibodies. ‘Epitope’ as used herein refers to that region of an antigenic polypeptide to which an antibody binds.

### Plasmid Vectors

Provided herein is a vector that is capable of expressing an antigen genes selected from MUC1, HER2, hTERT, Survivin, MAGEA3 and Mammaglobin A in the cell of a human in a quantity effective to elicit an immune response in the human. The vector may be a plasmid. The plasmid may be useful for transfecting cells with nucleic acid encoding these antigen genes, which the transformed host cell is cultured and maintained under conditions wherein expression of the malaria antigen takes place.

Plasmids may comprise DNA constructs which comprise one or more coding sequences encoding antigen genes selected from MUC1, HER2, hTERT, Survivin, MAGEA3 and Mammaglobin A as disclosed herein. The coding sequences encoding these genes as disclosed herein are preferable operably linked to regulatory elements. In some embodiments, a plasmid has DNA constructs that include coding sequence for three or more, four or more, five or more, or six antigen genes selected from MUC1, HER2, hTERT, Survivin, MAGEA3 and Mammaglobin A. In some embodiments, a plasmid has DNA constructs that include coding sequence for multiple antigen genes selected from MUC1, HER2, hTERT, Survivin, MAGEA3 and Mammaglobin A. In a plasmid having DNA constructs, these can include coding sequence for multiple antigen genes where the constructs may be separate expression cassettes wherein each antigen gene comprises a separate set of regulatory elements or two or more coding sequences may be incorporated into a single expression cassette in which coding sequences are separated by an IRS sequence.

Plasmids may comprise an initiation codon, which may be upstream of the coding sequence, and a stop codon, which may be downstream of the coding sequence. The initiation and termination codon may be in frame with the coding sequence. The plasmid may also comprise a promoter that is operably linked to the coding sequence The promoter operably linked to the coding sequence may be a promoter from simian virus 40 (SV40), a mouse mammary tumor virus (MMTV) promoter, a human immunodeficiency virus (HIV) promoter such as the bovine immunodeficiency virus (BIV) long terminal repeat (LTR) promoter, a Moloney virus promoter, an avian leukosis virus (ALV) promoter, a cytomegalovirus (CMV) promoter such as the CMV immediate early promoter, Epstein Ban-virus (EBV) promoter, or a Rous sarcoma virus (RSV) promoter. The promoter may also be a promoter from a human gene such as human actin, human myosin, human hemoglobin, or human muscle creatine. The promoter may also be a tissue specific promoter, such as a muscle or skin specific promoter, natural or synthetic. Examples of such promoters are described in U.S. Patent Application Publication No. 20040175727, the contents of which are incorporated herein in its entirety.

The plasmid may also comprise a polyadenylation signal, which may be downstream of the coding sequence. The polyadenylation signal may be a SV40 polyadenylation signal, LTR polyadenylation signal, bovine growth hormone (bGH) polyadenylation signal, human growth hormone (hGH) polyadenylation signal, or human β-globin polyadenylation signal. The SV40 polyadenylation signal may be a polyadenylation signal from a pCEP4 plasmid (Invitrogen). The plasmid may also comprise an enhancer upstream of the coding sequence. The enhancer may be human actin, human myosin, human hemoglobin, human muscle creatine or a viral enhancer such as one from CMV, FMDV, RSV or EBV.

The plasmid may also comprise a mammalian origin of replication in order to maintain the plasmid extrachromosomally and produce multiple copies of the plasmid in a cell. The plasmid may be pVAXI, pCEP4 or pREP4 (Invitrogen), which may comprise the Epstein Barr virus origin of replication and nuclear antigen EBNA-1 coding region, which may produce high copy episomal replication without integration. The backbone of the plasmid may be pAV0242. The plasmid may be a replication defective adenovirus type 5 (Ad5) plasmid. The plasmid may also comprise a regulatory sequence, which may be well suited for gene expression in a cell into which the plasmid is administered. The coding sequence for any antigen may comprise a codon that may allow more efficient transcription of the coding sequence in the host cell. Thus, the coding sequences may be codon optimized for more efficient translation.

The coding sequence may also comprise an Ig leader sequence. The leader sequence may be 5′ of the coding sequence. The consensus antigens encoded by this sequence may comprise an N-terminal Ig leader followed by a consensus antigen protein. The N-terminal Ig leader may be IgE or IgG. The plasmid may be pSE420 (Invitrogen), which may be used for protein production in Escherichia coli (E. coli). The plasmid may also be pYES2 (Invitrogen), which may be used for protein production in Saccharomyces cerevisiae strains of yeast. The plasmid may also be of the MAXBAC™ complete baculovirus expression system (Invitrogen), which may be used for protein production in insect cells. The plasmid may also be pcDNA I or pcDNA3 (Invitrogen), which may be used for protein production in mammalian cells such as Chinese hamster ovary (CHO) cells.

Immunogenic or vaccine compositions are provided which comprise plasmids. The immunogenic compositions may comprise a plurality of copies of a single nucleic acid molecule such a single plasmid, a plurality of copies of a two or more different nucleic acid molecules such as two or more different plasmids. For example, an immunogenic composition may comprise plurality of one, two, three, four, five, six, seven, eight, nine or ten or more different nucleic acid molecules. Such compositions may comprise plurality of one, two, three, four, five, six, seven, eight, nine or ten or more different plasmids. Compositions may comprise coding sequences for one or more of one or more coding sequences encoding antigen genes selected from MUC1, HER2, hTERT, Survivin, MAGEA3 and Mammaglobin A or antigenic fragments thereof. Immunogenic compositions may comprise nucleic acid molecules, such as plasmids, that collectively contain coding sequence for a single antigen gene selected from MUC1, HER2, hTERT, Survivin, MAGEA3 and Mammaglobin A or antigenic fragments thereof, coding sequence for two of these antigen genes, coding sequence for three of these antigen genes, coding sequence for four of these antigen genes, coding sequence for five antigens of these genes, or coding sequence for six of these antigen genes.

Compositions comprising coding sequence for antigen genes selected from MUC1, HER2, hTERT, Survivin, MAGEA3 and Mammaglobin A or antigenic fragments thereof may be on a single nucleic acid molecule such as a single plasmid or the compositions may comprise two different nucleic acid molecules such as two different plasmids wherein one nucleic acid molecule comprises the coding sequence one antigen gene and the other nucleic acid molecule comprises the coding sequence different antigen genes. Similarly, compositions comprising coding sequence three of these antigen genes may comprise a single nucleic acid molecule such as a single plasmid, two different nucleic acid molecules or three different nucleic acid molecules. Likewise, compositions comprising coding sequence four of these antigen genes may comprise a single nucleic acid molecule such as a single plasmid, two different nucleic acid molecules, three different nucleic acid molecules or four different nucleic acid molecules. Compositions comprising coding sequence five of these antigen genes may comprise a single nucleic acid molecule such as a single plasmid, two different nucleic acid molecules, three different nucleic acid molecules, four different nucleic acid molecules or five different nucleic acid molecules.

In some embodiments, a composition comprises a plurality single nucleic acid molecule encoding antigen genes selected from MUC1, HER2, hTERT, Survivin, MAGEA3 and Mammaglobin A or antigenic fragments thereof. In some embodiments, a composition comprises a plurality single nucleic acid molecules, such a single plasmid encoding two of these antigen genes or antigenic fragments thereof. In some embodiments, a composition comprises a plurality single nucleic acid molecules, such a single plasmid encoding three of these antigen genes or antigenic fragments thereof. In some embodiments, a composition comprises a plurality single nucleic acid molecules, such a single plasmid encoding four of these antigen genes or antigenic fragments thereof. In some embodiments, a composition comprises a plurality single nucleic acid molecules, such a single plasmid encoding five of these antigen genes or antigenic fragments thereof. In some embodiments, a composition comprises a plurality single nucleic acid molecules, such a single plasmid encoding six of these antigen genes or antigenic fragments thereof.

In some embodiments, a composition comprises a plurality two different nucleic acid molecules, such as two plasmids, each different nucleic acid molecule comprising a single different coding sequence for a different antigen genes selected from MUC1, HER2, hTERT, Survivin, MAGEA3 and Mammaglobin A or antigenic fragments thereof. Collectively, the two different plasmids encode two different antigen genes or antigenic fragments thereof. In some embodiments, a composition comprises a plurality two different nucleic acid molecules, such as two plasmids, which collectively comprise coding sequence for three different antigen genes. Collectively, the two different plasmids encode three different antigens or antigenic fragments thereof.

In some embodiments, a composition comprises a plurality two different nucleic acid molecules, such as two plasmids, collectively comprising coding sequence for four different antigen genes selected from MUC1, HER2, hTERT, Survivin, MAGEA3 and Mammaglobin A or antigenic fragments thereof. In some embodiments: one nucleic acid molecule encodes one these antigen genes or antigenic fragments thereof and the second encodes three different antigen genes or antigenic fragments thereof. Collectively, the two different plasmids encode four different antigen genes or antigenic fragments thereof.

In some embodiments, a composition comprises a plurality two different nucleic acid molecules, such as two plasmids, collectively comprising coding sequence for five different antigen genes selected from MUC1, HER2, hTERT, Survivin, MAGEA3 and Mammaglobin A or antigenic fragments thereof. In some embodiments, one nucleic acid molecule encodes two antigen genes or antigenic fragments thereof and the second encodes three antigen genes or fragments thereof. Collectively, the two different plasmids encode five different antigen genes or antigenic fragments thereof.

In some embodiments, a composition comprises a plurality two different nucleic acid molecules, such as two plasmids, collectively comprising coding sequence for six different antigen genes selected from MUC1, HER2, hTERT, Survivin, MAGEA3 and Mammaglobin A or antigenic fragments thereof. In some embodiments, one nucleic acid molecule encodes three antigen genes or antigenic fragments thereof and the second encodes three antigen genes or fragments thereof. Collectively, the two different plasmids encode six different antigen genes or antigenic fragments thereof.

### Modified Vaccinia Ankara Virus Vectors

In an embodiment, the present disclosure encompasses the use of recombinant modified vaccinia ankara (MVA) viruses for breast cancer immunization. The recombinant MVA are generated by insertion of heterologous sequences into an MVA virus. Examples of MVA virus strains that are useful in the practice of the present invention are strains MVA (as described in U.S. Pat. No. 5,185,146 herein incorporated by reference in its entirety, see also Altenburg (2014) Viruses 6, 2735-2761), MVA-572, MVA-575, MVA-BN and its derivatives, are additional exemplary strains. The coding sequence for MVA can be found at www.ncbi.nlm.nih.gov/nuccore/U94848.1. Although MVA is preferred for its higher safety (less replication competent), any MVA are suitable for this invention.

In certain embodiments, an MVA comprises three or more, four or more, five or more, or six genes encoding antigenic proteins or antigenic fragments thereof selected from MUC1, HER2, hTERT, Survivin, MAGEA3 and Mammaglobin A. In further embodiments, the antigen is not expressed in its full length form but is modified to be expressed as two or more fragments.

Such a breast cancer immunogenic agents are described herein in a non-limiting example and is referred to as “mvaBC.1” and “mvaBC.2” throughout the specification. As described herein, such immunogenic agents, including, but not limited to mvaBC.1 and mvaBC.2, are useful for the prophylactic immunization and treatment of breast cancer. The invention allows for the use of such agents in prime and boost vaccination regimens of humans, including patients at risk for breast cancer but not suffering from breast cancer, as well as those suffering from all stages of breast cancer. In an embodiment, the MVA is a boost immunization following an initial immunization with a plasmid DNA vector encoding three or more, four or more, five or more, or six genes encoding antigenic proteins or antigenic fragments thereof selected from MUC1, HER2, hTERT, Survivin, MAGEA3 and Mammaglobin A.

In certain embodiments, the MVA is MVA-BN, and is described in U.S. Pat. Nos. 6,761,893 and 6,193,752. In certain embodiments, a recombinant MVA is a derivative of MVA. Such “derivatives” include viruses that exhibit essentially the same replication characteristics as MVA, but that exhibit differences in one or more parts of its genome. Viruses that have the same “replication characteristics” as MVA are viruses that replicate with amplification ratios similar to MVA in CEF cells and HeLa, HaCat and 143B cell lines, and that show similar in vivo replication characteristics, as determined, for example, in the transgenic mouse model AGR129. In some embodiments, a MVA derivative is a codon-optimized version of MVA.

In certain embodiments, the MVA is a recombinant vaccinia virus that contains additional nucleotide sequences that are heterologous to the vaccinia virus. In certain of said embodiments, the heterologous sequences encode epitopes that induce a response by the immune system. Therefore, in certain embodiments, the recombinant MVA is used to vaccinate against proteins or agents comprising the epitope from any of MUC1, HER2, hTERT, Survivin, MAGEA3 and Mammaglobin A.

In certain embodiments, a heterologous nucleic acid sequence is inserted into a non-essential region of the virus genome. In certain of those embodiments, the heterologous nucleic acid sequence is inserted into a naturally occurring deletion site of the MVA genome. Methods for inserting heterologous sequences into the MVA genome are known to a person skilled in the art.

For vaccine preparation, MVA can be converted into a physiologically acceptable form. In certain embodiments, such a preparation is based on experience in preparing poxvirus vaccines used for smallpox vaccination.

For preparation, purified virus is stored at −80° C. with a titer of 5×108 TCID₅₀ per ml formulated in 10 mM Tris, 140 mM NaCl (pH 7.4). For vaccine dose preparation, for example, 10²-10⁸ virus particles can be lyophilized in phosphate buffered saline (PBS) in the presence of 2% peptone and 1% human albumin in a vial, preferably a glass vial. Alternatively, vaccine doses can be prepared in stages, lyophilization of the virus in a formulation. In certain embodiments, the formulation contains additional additives such as mannitol, dextran, sugar, glycine, lactose, polyvinyl pyrrolidone, or other additives such as, including, but not limited to antioxidants or inert gas, stabilizers or recombinant proteins (e.g., human serum albumin) suitable for in vivo administration. The vial is then sealed and can be stored at a suitable temperature, for example, between 4° C. and room temperature for several months. However, as long as there is no need, the vial is preferably stored at temperatures below −20° C.

In some embodiments involving vaccination or therapy, the lyophilisate is dissolved in 0.1 to 0.5 ml of an aqueous solution, preferably physiological saline or Tris buffer, and is administered systemically or locally, that is, parenterally, subcutaneous, intravenous, intramuscular, intranasal, intradermal, or any other route of administration known to a person skilled in the art. The optimization of the mode of administration, the dose and the number of administrations is within the skill and knowledge of a person skilled in the art.

### Methods of Immunization

Also described herein are methods for treating and/or preventing breast cancer, comprising administering to a subject in need thereof a composition comprising at least one vector encoding at least one of MUC1, HER2, hTERT, Survivin, MAGEA3 and Mammaglobin A, or antigenic fragments thereof, and the immunogen has an epitope that elicits a protective immune response, which is a humoral immune response (including, for example, a mucosal IgA, systemic IgA, IgG, IgM response) and/or a cell-mediated immune response, and pharmaceutically acceptable carrier, diluent, or excipient. The immunogen composition is administered at a dose sufficient to elicit an immune response specific for the administered vectors and variants thereof.

A human subject or host suitable for treatment with an immunogenic composition described herein may be identified by well-established indicators of risk for developing breast cancer or by well-established hallmarks of an existing disease. The immunogenic compositions that contain one or more vectors of the invention may be in any form that allows for the composition to be administered to a human subject. For example, a vector composition may be prepared and administered as a liquid solution or prepared as a solid form (e.g. lyophilized), which may be administered in solid form, or resuspended in a solution in conjunction with administration. The hybrid polypeptide composition is prepared or formulated to allow the active ingredients contained therein to be bioavailable upon administration of the composition to a subject or patient or to be bioavailable via slow release. Compositions that will be administered to a subject or patient take the form of one or more dosage units; for example, a tablet may be a single dosage unit, and a container of one or more compounds of the invention in aerosol form may hold a plurality of dosage units. In certain preferred embodiments, any of the aforementioned immunogenic compositions or vaccines comprising a vector of the invention are in a container, preferably in a sterile container.

In one embodiment, the immunogenic composition or vaccine is administered by parenteral means including intradermally or subcutaneously. The term “parenteral” as used herein, describes administration routes that bypass the gastrointestinal tract, including intradermal, intramuscular, intranasal, intraocular, intraperitoneal, intravenous, subcutaneous, submucosal, and intravaginal injection or infusion techniques. The term “transdermal/transmucosal” as used herein, is a route of administration in which the immunogenic composition is administered through or by way of the skin, including topical. The terms “nasal” and “inhalation” encompass techniques of administration in which an immunogenic composition is introduced into the pulmonary tree, including intrapulmonary or transpulmonary. In one embodiment, the compositions of the present invention are administered nasally.

In some embodiments, the immunogenic composition or vaccine contains an amount of plasmid vector from about 100 μg to about 1000 μg per dose. In some embodiments, the amount of vector administered per dose is from about 500 μg to about 700 μg. In some embodiments, the amount of vector administered per dose is from about 500 μg, 550 μg, 600 μg, 650 μg, 700 μg or more. In some embodiments, the amount of plasmid vector varies depending on dosing schedule. For example, an initial dose may be the same as, lower or higher than any subsequent immunization dose, including a booster immunization dose. The amount of plasmid administered per dose to any human subject can be adjusted according to the age, weight and physical condition of the human subject.

In some embodiments, the immunogenic composition or vaccine contains an amount of MVA vector from about 10³ to 10⁹ plaque forming units (pfu) per dose. In some embodiments, the amount of vector administered per dose is from about 10⁵ to 10⁷ pfu. In some embodiments, the amount of vector administered per dose is from about 10⁵ pfu, 10⁶ pfu, 10⁷ pfu or more. In some embodiments, the amount of MVA vector varies depending on dosing schedule. For example, an initial dose may be the same as, lower or higher than any subsequent immunization dose, including a booster immunization dose. The amount of plasmid administered per dose to any human subject can be adjusted according to the age, weight and physical condition of the human subject.

### Adjuvants

In some embodiments, the invention provides an immunogenic composition or vaccine comprising a vector encoding one or more MUC1, HER2, hTERT, Survivin, MAGEA3 and Mammaglobin A genes and a vaccine adjuvant. In one embodiment, a composition that is useful as an immunogenic composition for treating and/or preventing breast cancer contains at least one vector encoding MUC1, HER2, hTERT, Survivin, MAGEA3 and Mammaglobin A antigen (immunogen) as described herein capable of eliciting an immune response and recombinant human GM-CSF (also known as sargramostim). Sargramostim is a recombinant human granulocyte-macrophage colony stimulating factor (rhu GM-CSF) produced by recombinant DNA technology in a yeast (S. cerevisiae) expression system. GM-CSF is a hematopoietic growth factor which stimulates proliferation and differentiation of hematopoietic progenitor cells. Sargramostim is a glycoprotein of 127 amino acids characterized by three primary molecular species having molecular masses of 19,500, 16,800 and 15,500 daltons. The amino acid sequence of sargramostim differs from the natural human GM-CSF by a substitution of leucine at position 23, and the carbohydrate moiety may be different from the native protein. The liquid sargramostim is formulated as a sterile, preserved (1.1% benzyl alcohol), injectable solution (500 mcg/mL) in a vial. Lyophilized sargramostim is a sterile, white, preservative-free powder (250 mcg) that requires reconstitution with 1 mL Sterile Water for Injection, USP or 1 mL Bacteriostatic Water for Injection, USP. Liquid sargramostim has a pH range of 6.7-7.7 and lyophilized sargramostim has a pH range of 7.1-7.7. Liquid sargramostim and reconstituted lyophilized sargramostim are clear, colorless liquids suitable for subcutaneous injection (SC) or intravenous infusion (IV).

In some embodiments, the adjuvant is protollin or proteosome adjuvant (see e.g. U.S. Pat. No. 5,726,292). As is understood in the art, an adjuvant may enhance or improve the immunogenicity of an immunogen (that is, act as an immunostimulant), and many antigens are poorly immunogenic unless combined or admixed or mixed with an adjuvant. A variety of sources can be used as a source of antigen, such as live MVA encoding antigens, plasmids encoding antigens, split antigen preparations, subunit antigens, recombinant antigens, and combinations thereof. To maximize the effectiveness of a vector based vaccine, the vectors can be combined with a potent immunostimulant or adjuvant. Other exemplary adjuvants include alum (aluminum hydroxide, REHYDRAGEL); aluminum phosphate; virosomes; liposomes with and without Lipid A; or other oil in water emulsions type adjuvants such as MF-59 (Novartis), also such as nanoemulsions (see e.g. U.S. Pat. No. 5,716,637) or submicron emulsions (see e.g. U.S. Pat. No. 5,961,970); and Freund's complete and incomplete adjuvant.

A proteosome-based adjuvant (i.e. protollin or proteosome) can be used in vaccine compositions or formulations that may include any one or more of a variety of MUC1, HER2, hTERT, Survivin, MAGEA3 and Mammaglobin A antigen sources as described herein. Proteosomes are comprised of outer membrane proteins (OMP) from Neisseria species typically, but can be derived from other Gram-negative bacteria (see e.g. U.S. Pat. No. 5,726,292). Proteosomes have the capability to auto-assemble into vesicle or vesicle-like OMP clusters of 20-800 nm, and to noncovalently incorporate, coordinate, associate, or otherwise cooperate with protein antigens, particularly antigens that have a hydrophobic moiety. Proteosomes are hydrophobic, safe for human use, and comparable in size to certain viruses. By way of background, and not wishing to be bound by theory, mixing proteosomes with an antigen such as a protein antigen, provides a composition comprising non-covalent association or coordination between the antigen and proteosomes, which association or coordination forms when solubilizing detergent is selectively removed or reduced in concentration, for example, by dialysis.

Any preparation method that results in the outer membrane protein component in vesicular or vesicle-like form, including molten globular-like OMP compositions of one or more OMP, is included within the scope of proteosome. In one embodiment, the proteosomes are from Neisseria species, and from Neisseria meningitidis. In certain other embodiments, proteosomes may be an adjuvant and an antigen delivery composition. In an embodiment, an immunogenic composition comprises one or more vectors encoding a breast cancer antigen and an adjuvant, wherein the adjuvant comprises Projuvant or Protollin. In certain embodiments, an immunogenic composition further comprises a second immunostimulant, such as a liposaccharide. That is, the adjuvant may be prepared to include an additional immunostimulant. For example, the projuvant may be mixed with a liposaccharide to provide an OMP-LPS adjuvant. Thus, the OMP-LPS (protollin) adjuvant can be comprised of two components. The first component includes an outer membrane protein preparation of proteosomes (i.e. Projuvant) prepared from Gram-negative bacteria, such as Neisseria meningitidis, and the second component includes a preparation of liposaccharide. It is also contemplated that the second component may include lipids, glycolipids, glycoproteins, small molecules or the like, and combinations thereof. As described herein, the two components of an OMP-LPS adjuvant may be combined (admixed or formulated) at specific initial ratios to optimize interaction between the components, resulting in stable association and formulation of the components for use in the preparation of an immunogenic composition. The process generally involves the mixing of components in a selected detergent solution (e.g. Empigen BB, Triton x-100 or Mega-10) and then effecting complex formation of the OMP and LPS components while reducing the amount of detergent to a predetermined, preferred concentration by dialysis or by diafiltration/ultrafiltration methodologies. Mixing, co-precipitation, or lyophilization of the two components may also be used to effect an adequate and stable association, composition, or formulation. In one embodiment, an immunogenic composition comprises one or more vectors encoding MUC1, HER2, hTERT, Survivin, MAGEA3 and Mammaglobin A antigens and an adjuvant, wherein the adjuvant comprises a projuvant (i.e. proteosome) and liposaccharide.

In an embodiment, the final liposaccharide content by weight as a percentage of the total proteosome protein can be in a range from about 1% to about 500%, also in range from about 10% to about 200%, or in a range from about 30% to about 150%. Another embodiment includes an adjuvant wherein the proteosomes are prepared from Neisseria meningitidis and the liposaccharide is prepared from Shigella flexneri or Plesiomonas shigelloides, and the final liposaccharide content is between 50% to 150% of the total Proteosome protein by weight. In another embodiment, proteosomes are prepared with endogenous lipooligosaccharide (LOS) content ranging from about 0.5% up to about 5% of total OMP. In another embodiment proteosomes have endogenous liposaccharide in a range from about 12% to about 25%, and in still another embodiment the endogenous liposaccharide is between about 15% and about 20% of total OMP. The instant disclosure also provides an immunogenic composition containing liposaccharide derived from any Gram-negative bacterial species, which may be from the same Gram-negative bacterial species that is the source of proteosomes or may be from a different bacterial species. In certain embodiments, the proteosome or protollin to vector ratio in the immunogenic composition is greater than 1:1, greater than 2:1, greater than 3:1 or greater than 4:1. In other embodiments, proteosome or protollin to vector ratio in the immunogenic composition is about 1:1, 2:1, 3:1 or 4:1. The ratio can be 8:1 or higher. In other embodiments, the ratio of proteosome or protollin to vector in the immunogenic composition ranges from about 1:1 to about 1:500, and is at least 1:5, at least 1:10, at least 1:20, at least 1:50, or at least 1:100, or at least 1:200.

In other embodiments, immunogenic compositions may comprise (projuvant or protollin), or further comprise components (e.g. receptor ligands) capable of stimulating a host immune response by interacting with certain receptors (e.g. Toll-like receptors or “TLR”) produced by one or more host cells of a vaccine recipient. According to one embodiment, compositions comprising immunogenic epitopes of a MUC1, HER2, hTERT, Survivin, MAGEA3 and Mammaglobin A protein may contain polypeptide epitopes capable of interacting with Toll-like receptors, thereby promoting an innate immune response, which may or may not evoke a subsequent adaptive immune response.

An innate immune response is a nonspecific protective immune response that is not a specific antigen-dependent or antibody-dependent response (that is, does not involve clonal expansion of T cells and/or B cells) and may be elicited by any one of numerous breast cancer antigens or immunogens as described herein. An immunostimulatory composition described herein comprises proteosomes and liposaccharide (protollin), either one of which or both may elicit a nonspecific protective response. Without wishing to be bound by theory, one or more components of vaccine compositions or formulations disclosed herein may interact with Toll-like receptors associated with an innate or adaptive immune response of a vaccine recipient. One or more ligands that interact with and subsequently activate certain TLR have been identified, with the exception of TLR8 and TLR10. Certain outer membrane proteins of Neisseria meningitidis, for example OMP2 (also referred to as PorB), interact with TLR2, while LPS of most but not all Gram-negative bacteria interacts with TLR4. Accordingly, one activity of vaccine compositions or formulations described herein, which may contribute to a biological effect, includes activation of one or both of TLR2 and TLR4. Activation of other TLR (other than TLR2 and TLR4) may serve a similar function or further enhance the qualitative or quantitative profile of cytokines expressed, and may be associated with a host Th1/Th2 immune response. It is also contemplated that TLR ligands other than LPS and PorB may be used alone or in combination to activate TLR2 or TLR4. The qualitative or quantitative activation of one or more TLR is expected to elicit, effect, or influence a relative stimulation (balanced or unbalanced) of a Th1 or Th2 immune response, with or without a concomitant humoral antibody response. Ligands interacting with TLR other than TLR2 and TLR4 may also be used in vaccine compositions described herein. Such vaccine components may, alone or in combination, be used to influence the development of a host immune response sufficient to treat or protect from virus infection, as set forth herein.

Other components known to the art may be used in the compositions described herein. Some embodiments of the immunogen may further comprise adjuvants, such as Bacillus Calmette-Guérin (BCG), cytokines (for non-limiting example, granulocyte-macrophage colony-stimulating (GM-CSF)), aluminum gels or aluminum salts, or other adjuvants known to the art to non-specifically stimulate immune response and enhance the efficacy of the immune response to the vaccine. In at least one preferred embodiment, the adjuvant is BCG Tice.

An immunogenic composition or vaccine may further comprise preservatives known to the art, including without limitation, formaldehyde, antibiotics, monosodium glutamate, 2-phenoxyethanol, phenol, and benzethonium chloride. An immunogenic composition or vaccine may further comprise sterile water for injection, balanced salt solutions for injections.

While some embodiments of the invention are shown and described herein, such embodiments are provided by way of example only and are not intended to otherwise limit the scope of the invention. Various alternatives to the described embodiments of the invention may be employed in practicing the invention. Therefore, the spirit and scope of the appended claims should not be limited to the description of the preferred versions contained herein.

The attention of the reader is directed to all papers and documents which are filed concurrently with this specification and which are open to public inspection with this specification, and the contents of all such papers and documents incorporated herein by reference. All the features disclosed in the specification (including any accompanying claims, abstract, and drawings) may be replaced by alternative features serving the same, equivalent or similar purpose, unless expressly stated otherwise. Thus, unless expressly stated otherwise, each feature disclosed is one example only of a generic series of equivalent or similar features.

### EXAMPLES

### Example 1: Vaccine Preparation

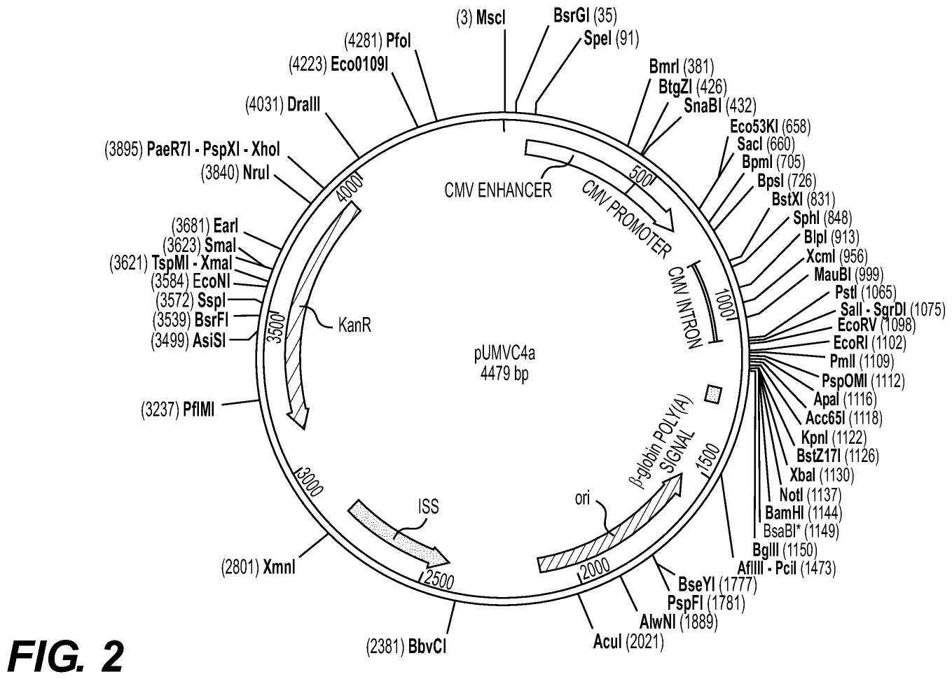

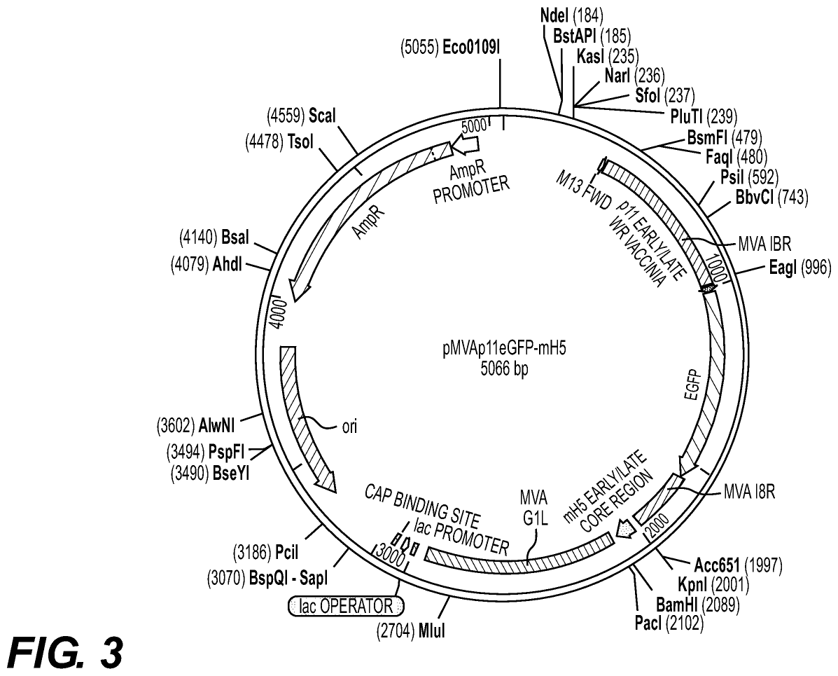

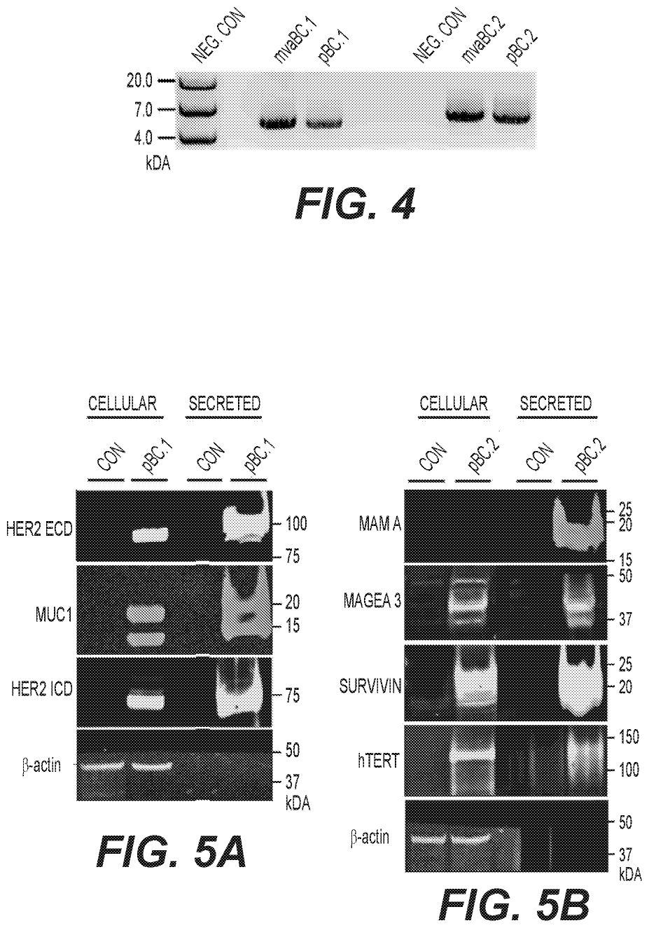

Vaccines to co-express the six antigens as a product of two constructs using 2A peptide technology were designed and produced (FIG. 1). HER2 was split to prevent oncogenic activity associated with the full-length protein. Additionally, distinct signal sequences (SS) were added to the N-terminus of antigens with known subcellular expressions (HER2 ICD, hTERT, MAGEA3, and Survivin) to promote extracellular secretion. The multi-antigen gene cassettes were inserted into pUMVC4a vaccine plasmid (FIG. 2) to create, pBC.1 and pBC.2, and their expression validated by transient transfection in HEK293 cells. All antigens are expressed in cellular and secreted fractions, except for Mammaglobin A which was exclusively secreted (FIG. 2). To generate recombinant MVA, the two multi-antigen gene cassettes (FIG. 1) were codon optimized for Vaccinia virus and inserted into the pMVApIleGFP-mH5 transfer vector (FIG. 2) to create mvaBC.1 and mvaBC.2. Generation and amplification of MVA virus was done by the University of Iowa Viral Vector Core Facility. After performing final QC by PCR/Sequencing (Suppl. Data 4), recombinant expression of target antigens from mvaBC.1 and mvaBC.2 were evaluated by infection of DF-1 cells. Expression patterns of the individual antigens was consistent with that of plasmid based expression (FIG. 2), being expressed in the cell and extracellularly (FIG. 3). All plasmids were verified by next-generation sequencing and MVA through targeted sequencing.

### Example 2: In Vitro Expression of Target Antigens from Plasmid Vaccines

HEK293 cells were transfected with plasmids pBC.1 and pBC.2. Following incubation in serum free media for 24 hours, cell culture supernatants were precipitated with acetone and cellular lysates prepared. Twenty micrograms of cellular lysates and 20 μl of supernatants (out of 100 μl) were analyzed by SDS-PAGE Western blot using antigen specific antibodies. FIG. 5 represents Western blot analysis of cellular and secreted (supernatant) fractions from pBC.1 and pBC.2 plasmid transfected HEK293 cells. Membranes were probed with a 1:1000 dilution of antibodies against Her2 ECD (extracellular domain), Her2 ICD (intracellular domain), Magea3, Survivin and β-actin. Antibodies against Mucin-1 and hTert were used at 1:50 and 1:500 dilutions, respectively. Detection was performed using appropriate IRDye antibodies (1:5000 dilution) and detected using the LicoR Odyssey CLx.

### Example 3: In Vitro Expression of Target Antigens from Recombinant MVA

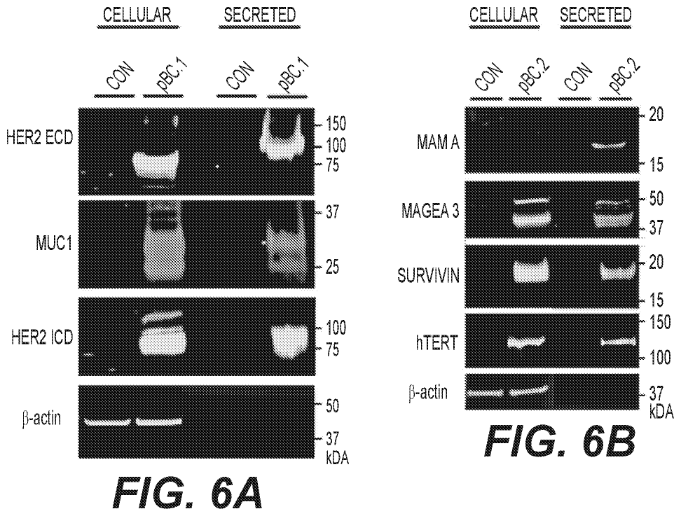

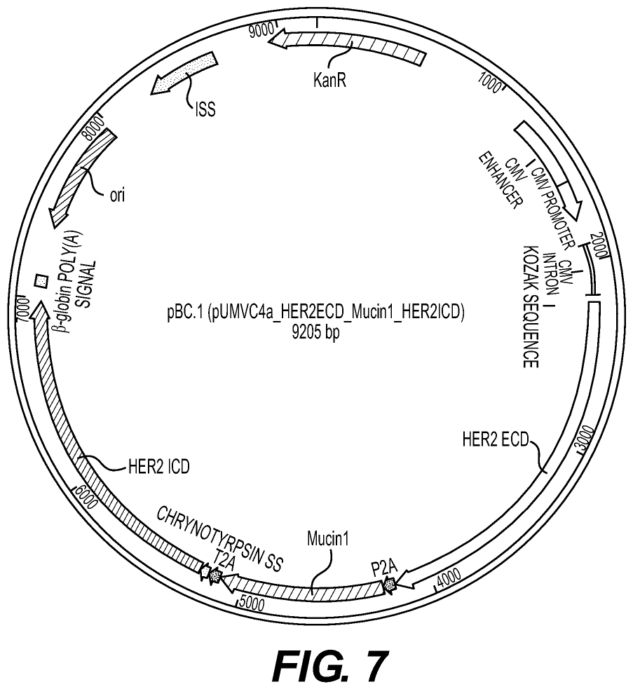

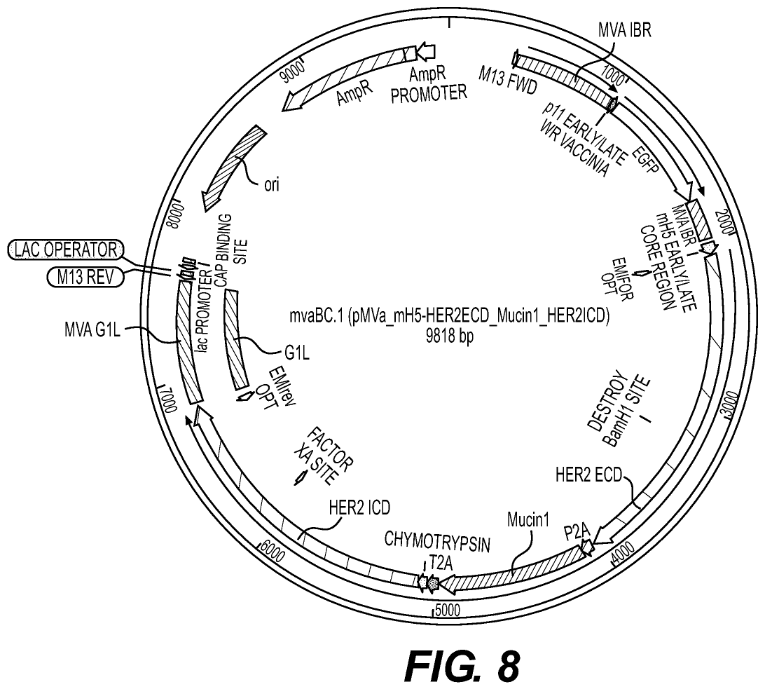

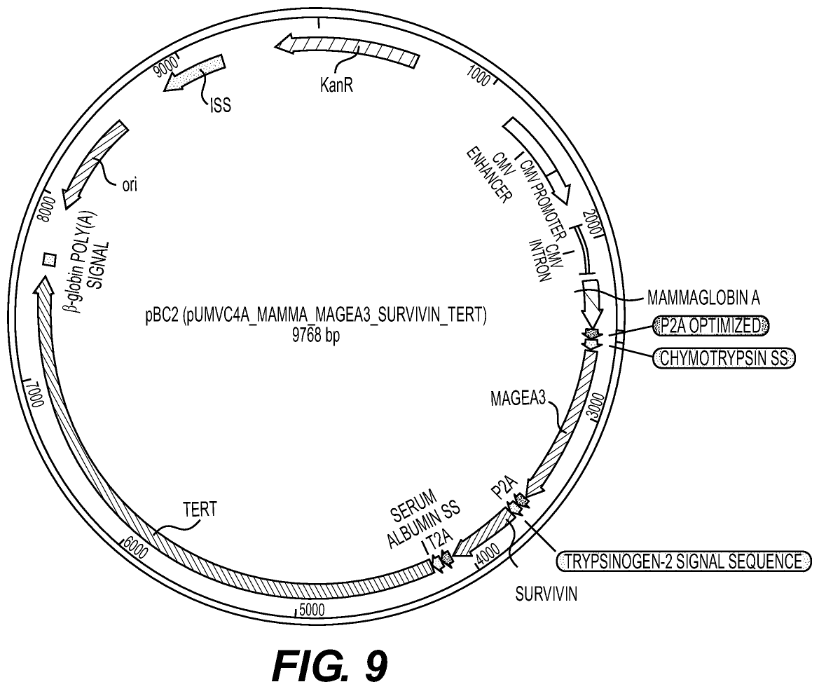

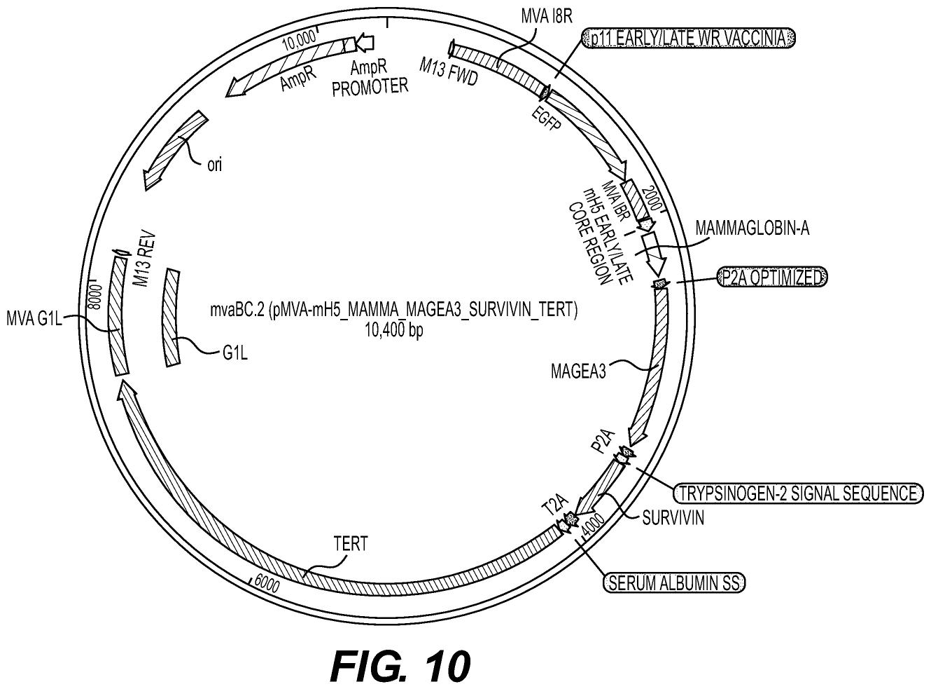

DF-1 cells were infected with mvaBC.1 and mvaBC.2 at a multiplicity of infection (MOI) of 5 for two hours. Cells were then allowed to recover overnight and then incubated in serum free media for 24 hours. Cell culture supernatants were precipitated with acetone and cellular lysates were prepared. Twenty micrograms of cellular lysates and 20 μl of supernatants (out of 100 μl) were analyzed by SDS-PAGE Western blot using antigen specific antibodies. FIG. 6 represents Western blot analysis of cellular and secreted (supernatant) fractions from mvaBC.1 and mvaBC.2 infected DF-1 cells. Membranes were probed with a 1:1000 dilution of antibodies against Her2 ECD (extracellular domain), Her2 ICD (intracellular domain), Magea3, Survivin and β-actin. Antibodies against Mucin-1 and hTert were used at 1:50 and 1:500 dilutions, respectively. Detection was performed using appropriate IRDye antibodies (1:5000 dilution) and detected using the LicoR Odyssey CLx.

### Example 4: Clinical Study

The present clinical protocol proposes to test a vaccine strategy that will enable recipients to boost internal immune defenses against breast cancer. Unlike infectious disease vaccines, however, the proposed vaccine targets self-antigens which have low level expression in normal healthy tissue, but high level expression in tumors. The vaccine targets six commonly upregulated antigens, MUC1, HER2, hTERT, Survivin, MAGEA3 and Mammaglobin A. Founded on an extensive backdrop of recent preclinical and clinical supporting data, the present vaccine is designed to safely generate biologically relevant immunity with the intent of preventing development of the three major subsets of breast cancer, ER+, HER2+ and triple negative breast cancer. The vaccination process will consist of two interventions in healthy people at risk for developing breast cancer, an initial priming intervention with an equimolar mixture of 6 plasmids followed by a boost at 30 days after the priming with an equimolar mixture of six Modified Vaccine Ankara Virus constructs, each expressing one of the antigens. Safety of administration and immunogenicity of the approach will be examined over the course of 3 months following trial registration. The ultimate goal is to establish a safe prevention vaccine for all forms of breast cancer.

The present clinical protocol employs a novel multi-antigen plasmid DNA-prime MVA-boost vaccine. This particular vaccine is composed of two multi-antigen plasmid DNA vaccines followed by boosting with modified vaccinia virus Ankara (MVA) containing similar multi-antigen plasmid DNA 30±3 days after the first vaccination. This particular DNA-prime MVA-boost strategy has been shown to be safe and able to induce more potent cellular immune response. In HIV vaccine development, a heterologous DNA prime-MVA boost regimen has been shown to generate significant improved T cell responses compared to homologous vaccination with either the DNA or MVA vaccine alone (van Diepen et al. (2019) J. Virol. 93: e02155-18). The first two multi-antigen plasmid DNA vaccines include Artemis 1.P1 and Artemis 1.P2. The plasmid constructs are as shown in FIG. 1.

A plasmid DNA dose (4 mg total/2 mg each construct) is to be administered. Modified Vaccinia virus Ankara (MVA) is derived from Chorioallantois Vaccinia virus Ankara by serial passaging in chicken embryo fibroblasts (Altenburg et al. (2014) Viruses 6, 2735-2761). It has been established that 1×10⁸ plaque-forming units is an immunogenic and safe dose regardless of the antigen (Buchbinder et al. (2017) PLoS One 12, e0179597). For the current study, recombinant human GM-CSF (rhuGM-CSF) will be used at a total injection dose of 125 mcg admixed prior to injection with peptide and injected intradermally at the time of immunization. Local effects at the injection site are not expected with this dose of GM-CSF. rhuGM-CSF is generally well tolerated when administered intravenously or subcutaneously in doses ranging from 50-500 mcg/m²/day.

The primary goal is to determine the safety and tolerability of a multi-antigen plasmid DNA-prime MVA-boost vaccine, Artemis 1, in patients with metastatic breast cancer. The ability of Artemis 1 to elicit an immune response as measured will also be determined by high-affinity antibodies against HER2, MUC1, Mammoglobin A, Survivin, hTERT, and MAGEA3. Secondary goals include assessment of the objective response rate (ORR) and clinical benefit rate (CBR) after two doses of vaccine and assessment of the ORR and CBR in subsequent therapy after vaccination. The ability of the expressed HER-2/neu peptide 885 to generate a T cell response that is specific to HER-2/neu or is cross-reactive with EGFR protein will be determined. The presence of HLA-DR epitopes that contain HLA Class I embedded epitopes will also be assessed.

Inclusion criteria. Female age >18 years. Histological confirmation of adenocarcinoma of the breast with unresectable locally advanced or metastatic disease and have all of the following: Low disease burden as per investigator discretion. Suitable for vaccinations and follow up of 60 days without chemotherapy (note: endocrine therapies including tamoxifen, ovarian suppression, aromatase inhibitors (anastrozole, letrozole, and exemestane), and fulvestrant are permitted during the vaccinations. Life expectancy more than 6 months. No standard curative therapy option.

Treatment Schedule. Study Treatment will be administered as outlined in the tables below. Patients will receive a total of 2 vaccinations with 30±3 days apart. After completion of vaccinations, patients will be followed for 30 days post-vaccinations (note: Concurrent uses of endocrine therapies, such as aromatase inhibitors (letrozole, anastrozole, or exemestane), fulvestrant, ovarian suppressions, or tamoxifen are permitted during the trial). The Artemis 1 vaccine will be injected intra-dermally. The first 6 patients in the safety lead in will be treated with Artemis 1.P1 with GM-CSF during the first vaccination followed by Artemis 1.V1 30±3 days after the first injection.

i. Vaccination in Safety Lead In Study

| Agent | Dose Level | Route | Treatment Day |
|---|---|---|---|
| Artemis 1.P1 + | 600 μg, | Intradermal | Day 1 |
| Adjuvant | admixed with | injection | |
| (GM-CSF) | 125 μg of | | |
| | GM-CSF | | |
| Artemis 1.V1 | 10⁸ plaque | Intradermal | Day 30 |
| | forming | injection | (±3 days) |
| | units (pfu) | | |

If there is no significant DLT observed in the first 6 patients, 19 additional patients will receive Artemis 1.P1+Artemis 1.P2 with GM-CSF during the first vaccination followed by Artemis 1.V1+Artemis 1.V2 30±3 days after the first injection.

ii. Vaccination in Study

| Agent | Dose Level | Route | Treatment Day |
|---|---|---|---|
| Artemis 1.P1 + | 600 μg | Intradermal | Day 1 |
| Artemis 1.P2 + | each, admixed | injection | |
| Adjuvant | with 125 μg | | |
| (GM-CSF) | of GM-CSF | | |
| Artemis 1.V1 + | 10⁸ pfu each | Intradermal | Day 30 ± |
| Artemis 1.V2 | | injection | 3 days |

Treatment Evaluation & Measurement of Effect. Response and progression will be evaluated in this study using the new international criteria proposed by the revised Response Evaluation Criteria in Solid Tumors (RECIST) guidelines (version 1.1) and by the ESMO 2014 Adaptation of the Immune-Related Response Criteria: irRECIST (Seymour et al. (2017) Lancet. Oncol. 18, e143-e152). RECIST version 1.1 will be used for assessment of tumor response for the primary endpoint.

Changes in the largest diameter (unidimensional measurement) of the tumor lesions and the short axis measurements in the case of lymph nodes are used in the RECIST 1.1 and irRECIST guidelines. In RECIST 1.1, the appearance of new lesions automatically signifies progressive disease (PD), while in irRECIST new measurable lesions are factored into the total tumor burden. The specifics are presented below.

Schedule of Evaluations. For the purposes of this study, patients should be reevaluated every 6 weeks. In addition to a baseline scan, confirmatory scans should also be obtained 4 weeks following initial documentation of objective response.

Measurable Disease. A non-nodal lesion is considered measurable if its longest diameter can be accurately measured as >2.0 cm with chest x-ray, or as 2′1.0 cm with CT scan, or MRI. A superficial non-nodal lesion is measurable if its longest diameter is 2′1.0 cm in diameter as assessed using calipers (e.g. skin nodules) or imaging. In the case of skin lesions, documentation by color photography, including a ruler to estimate the size of the lesion, is recommended. A malignant lymph node is considered measurable if its short axis is >1.5 cm when assessed by CT scan (CT scan slice thickness recommended to be no greater than 5 mm).

Non-Measurable Disease. All other lesions (or sites of disease) are considered non-measurable disease, including pathological nodes (those with a short axis 2′1.0 to <1.5 cm). Bone lesions, leptomeningeal disease, ascites, pleural/pericardial effusions, lymphangitis cutis/pulmonis, inflammatory breast disease, and abdominal masses (not followed by CT or MRI), are considered as non-measurable as well.

Administration. Artemis 1.P1 and Artemis 1.P2: Administer the plasmid vaccine to the patient as soon as possible after preparation, labeling and dose confirmation. A Biojector® 2000 will be used to deliver the plasmid vaccine. Injection should be intramuscularly (I.M.) in the deltoid muscle in either of the upper extremity. However, the upper extremity without previous axillary lymph node dissection is the preferred side. Artemis 1.V1 and Artemis 1.V2: Administer the MVA vaccine to the patient as soon as possible after preparation, labeling and dose confirmation. The MVA vaccine will be injected intramuscularly (I.M.) at one site in the deltoid muscle in either of the upper extremity. However, the upper extremity without previous axillary lymph node dissection is the preferred side. GM-CSF will be mixed and given at the same time with plasmid vaccine Artemis 1.P1 and Artemis 1.P2.

1. An immunogenic composition comprising a vector encoding at least one epitope from at least three antigens encoded by a gene selected from the group consisting of MUC1, HER2, hTERT, Survivin, MAGEA3 and Mammaglobin A, and at least one pharmaceutically acceptable excipient.

2. The immunogenic composition of claim 1, wherein the vector encodes at least four antigens encoded by a gene selected from the group consisting of MUC1, HER2, hTERT, Survivin, MAGEA3 and Mammaglobin A.

3. The immunogenic composition of claim 1, wherein the vector encodes at least five antigens encoded by a gene selected from the group consisting of MUC1, HER2, hTERT, Survivin, MAGEA3 and Mammaglobin A.

4. The immunogenic composition of claim 1, wherein the vector encodes at least six antigens encoded by a gene selected from the group consisting of MUC1, HER2, hTERT, Survivin, MAGEA3 and Mammaglobin A.

5. The immunogenic composition of claim 1, wherein the at least one epitope encoded by the MUC1 gene comprises the full length protein encoded by the MUC1 gene.

6. The immunogenic composition of claim 1, wherein the at least one epitope encoded by the HER2 gene comprises amino acids 1 to 652 of the protein encoded by the HER2 gene.

7. The immunogenic composition of claim 1, wherein the at least one epitope encoded by the HER2 gene comprises amino acids 676 to 1254 of the protein encoded by the HER2 gene.

8. The immunogenic composition of claim 1, wherein the at least one epitope encoded by the hTERT gene comprises amino acids 15 to 1132 of the protein encoded by the hTERT gene.

9. The immunogenic composition of claim 1, wherein the at least one epitope encoded by the MAGEA3 gene comprises the full length protein encoded by the MAGEA3 gene.

10. The immunogenic composition of claim 1, wherein the at least one epitope encoded by the Survivin gene comprises the full length protein encoded by the Survivin gene.

11. The immunogenic composition of claim 1, wherein each epitope is separated by a cleavable spacer sequence.

12. The immunogenic composition of claim 1, wherein the vector is a DNA vector.

13. The immunogenic composition of claim 1, wherein the vector is a Modified Vaccinia Ankara (MVA) virus.

14. A method of inducing an antibody-mediated or T cell mediated immune response in a human subject against one or more antigens selected from the group consisting of MUC1, HER2, hTERT, Survivin, MAGEA3 and Mammaglobin A comprising administering to the human subject the immunogenic composition of claim 1.

15-18. (canceled)

19. The method of claim 14, wherein the human subject is not suffering from breast cancer.

20. The method of claim 14, wherein the human subject is suffering from primary breast cancer.

21. The method of claim 14, wherein the human subject is suffering from metastatic breast cancer.

22. The method of claim 14, wherein the human subject is suffering from Stage 0, I, II, III or IV breast cancer.

23. The method of claim 14, wherein the human subject is not receiving chemotherapy.
